(19) United States
(12) Patent Application Publication
(10) Pub. No.: US 2026/0097029 A1
(43) Pub. Date: Apr. 9, 2026

(54) Tetrahydroquinoline Compounds As Antitumor Agents

(71) Applicants: Sandro COSCONATI, Caserta (IT); Stefano TOMASSI, Roma (IT); Salvatore DI MARO, Napoli (IT); Hilda Amelia PICKETT, Surry Hills, New South Wales (AU)

(72) Inventors: Sandro COSCONATI, Caserta (IT); Stefano TOMASSI, Roma (IT); Salvatore DI MARO, Napoli (IT); Hilda Amelia PICKETT, Surry Hills, New South Wales (AU)

(21) Appl. No.: 19/112,485

(22) PCT Filed: Sep. 22, 2023
(86) PCT No.: PCT/EP2023/076229

§ 371 Date: Mar. 17, 2025

(30) Foreign Application Priority Data
Sep. 26, 2022 (IT) 102022000019713

(51) Int. Cl. A61K 31/4709; A61K 31/47; A61P 35/00; C07D 215/06; C07D 401/12; C07D 405/12

(52) U.S. Cl. CPC A61K 31/4709; A61K 31/47; A61P 35/00; C07D 215/06; C07D 401/12; C07D 405/12

(57) ABSTRACT

The invention relates to a tetrahydroquinoline compound of Formula (I) or a pharmaceutically acceptable salt thereof wherein A is (C₃-C₇)cycloalkyl; (C₃-C₇)heterocycloalkyl comprising from 1 to 3 heteroatoms selected from O, N, and S; (C₁-C₇)alkyl, (C₁-C₇) heteroalkyl comprising from 1 to 3 heteroatoms selected from O, N, and S; an optionally substituted phenyl ring or an optionally substituted (5-9)-membered heteroaromatic ring comprising from 1 to 3 heteroatoms selected from O, N, and S; R is H, (C₁-C₄) alkyl; (C₁-C₃)alkoxy(C₁-C₃)alkyl, (C₃-C₇)cycloalkyl; R₁ and R₂ are, independently from each other, selected from the group consisting of H, NO₂, CN, halogen, (C₁-C₄)alkyl; (C₂-C₄)alkenyl; (C₁-C₄)alkoxy(C₁-C₄)alkyl, (C₃-C₇)cycloalkyl(C₁-C₄)alkyl, (C₁-C₄)alkyl substituted by a (5-9)-membered heteroaromatic ring comprising from 1 to 3 heteroatoms selected from O, N, and S, amino optionally substituted with bis(dimetilamino)methylene, pyridyl, trifluoromethyl-SO₂—, (C₁-C₃)alkyl-SO₂—, a group NH—CO—R₃, wherein R₃ is selected from the group consisting of benzyl, (C₃-C₇)cycloalkyl(C₁-C₄)alkyl, phenyl, vinyl, halogen-CH₂—, (C₁-C₃)alkyl; a group NH—CH₂—R₄ wherein R₄ is an optionally substituted phenyl ring or an optionally substituted (5-9)-membered heteroaromatic ring comprising from 1 to 3 heteroatoms selected from O, N and S; or R₁ and R₂ together form an optionally substituted aromatic ring comprising 6 carbon atoms. The tetrahydroquinoline compound of or a pharmaceutically acceptable salt thereof according to the invention is a disruptor of the FANCM/BTR interaction so as to hamper the FANCM localization to telomeres, preferably in the treatment of tumours.

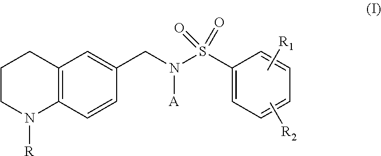

### FIELD OF THE INVENTION

The present invention relates to new antitumor agents.

### BACKGROUND

The alternative lengthening of telomeres (ALT) is a mechanism of telomere maintenance that is observed in many of the most recalcitrant cancer subtypes. In ALT, telomeres are maintained through the implication of multiple homology-directed repair (HDR) mechanisms that allow for DNA extension. A wealth of data demonstrates that ALT elongated telomeres feature a permissive chromatin state, stochastic DNA damage, and an augmented level of replication stress. This latter can be ascribed to the presence of variant repeats (e.g. TCAGGG, TGAGGG) throughout ALT telomeres resulting from recombination-mediated telomere replication.1 Moreover, replication stress in ALT cancers has been connected with a distinctive nucleoprotein architecture shaped by the mismanagement of chromatin that fosters cycles of DNA damage.2

In general, telomeres pose a challenge for the replication machinery, leading to replication fork stalling. Fork reversal and restart (mediated by FANCM3 and SMARCAL1)4 relieves replication stress at telomeres, resulting in the restriction of ALT activity. Conversely, unresolved replication stress leads to fork collapse providing DSBs substrates for ALT-mediated recombination and telomere synthesis. Thus, proteins implicated in ameliorating the replication stress at telomeres are intrinsically involved in ALT regulation.

ALT is often found in cancers of a mesenchymal origin, including central nervous system cancers as well as in many sarcomas, particularly osteosarcoma (OS).5 This might be explained by the fact that telomerase expression in mesenchymal cells is more stringently controlled compared to epithelial cells.4 The prevalence of ALT cancers (10-15%) is underestimated considering the deficiencies in direct clinical diagnostics for ALT detection. In addition to OS (64% prevalence), pancreatic neuroendocrine (PanNETs), and soft tissue tumors such as leiomyosarcoma, ALT is also prevalent in low-grade astrocytoma and secondary glioma, as well as in several pediatric cancers such as neuroblastoma, diffuse intrinsic pontine glioma (DIPG), and glioblastoma.2 To date, ALT activity has not been detected in normal cells, underscoring a distinctive cancer-selective therapeutic opportunity. However, despite this selectively cancer-associated feature, effective drugs that selectively kill ALT-positive cancer cells are yet to be discovered.

OS is the best example of ALT cancer. It is the most common type of primary malignant bone tumor, which is defined by the presence of malignant mesenchymal cells producing osteoid or immature bone. It accounts for 30%-80% of the primary skeletal sarcomas. The population affected is predominantly children, teenagers, and young adults aged 10-30 years.6 Males are more affected than females. The peak incidence of the most frequent type of OS, i.e. high-grade central OS, is occurring in the second decade of life during the adolescent growth spurt. If left untreated, OS progresses locally and systemically and leads to death within months. The outcome for patients with OS was poor before the use of effective chemotherapy, with 2-year overall survival rates of 15%-20% following surgical resection and/or radiotherapy. Neoadjuvant chemotherapy (involving high-dose methotrexate with leucovorin rescue, doxorubicin, cisplatin, bleomycin, cyclophosphamide, and dactinomycin) followed by definitive surgery has the advantage of facilitating limb salvage procedures. Almost all patients have microscopic metastases at the time of diagnosis, as evidenced by the fact that 80%-90% develop metastatic recurrence if treated with surgical resection and/or radiotherapy. These patients have an especially poor prognosis with a five-year survival rate of only 20-30% compared to 60-66% for patients with localized disease.7

The discoveries described in the present document are based on two major discoveries made by the research team. First, in 2019 the Pickett's laboratory has identified an Achilles' heel of ALT cells, allowing to induce for the first time ALT-specific cell death. This was achieved through the induction of rampant replication stress at telomeres. They demonstrated that depletion of FANCM, a DNA translocase that functionally interacts with the BLM-TOP3A-RMI (BTR) complex and the Fanconi anemia (FA) core complex, resolves replication stress that normally occurs at telomeres. ALT telomeres feature distinctive structural aberrations that prompt replication defects and are therefore hypersensitive to FANCM depletion. In the absence of FANCM, stalled forks deteriorate to form DSBs. These data demonstrate proof of concept of the toxic effects of exacerbated levels of telomere replication stress to ALT cells and implicate the FANCM/BTR system as a promising target for the treatment of ALT cancers. Therefore, the patent publication WO2020/242330 describes a method for inhibiting FANCM activity or expression, such as inhibiting FANCM's interaction with RMI and/or FANCM's ATPase activity, to inhibit the growth and/or proliferation of ALT tumor cells and/or to induce death of ATL tumor cells.

The object of the present invention is to provide disruptors of the FANCM/BTR interaction so as to hamper the FANCM localization to telomeres and so to treat tumours.

### SUMMARY OF THE INVENTION

The inventors surprisingly found out the chemical disruptors of the FANCM/BTR interaction so as to hamper the FANCM localization to telomeres.

Therefore, the present invention concerns a tetrahydroquinoline compound of Formula (I) or a pharmaceutically acceptable salt thereof

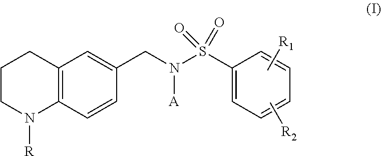

wherein
A is (C₃-C₇)cycloalkyl; (C₃-C₇)heterocycloalkyl comprising from 1 to 3 heteroatoms selected from O, N, and S; (C₁-C₇)alkyl, (C₁-C₇)heteroalkyl comprising from 1 to 3 heteroatoms selected from O, N, and S; an optionally substituted phenyl ring or an optionally substituted (5-9)-membered heteroaromatic ring comprising from 1 to 3 heteroatoms selected from O, N, and S;
R is H, (C₁-C₄)alkyl; (C₁-C₃)alkoxy(C₁-C₃)alkyl, (C₃-C₇)cycloalkyl;
R₁ and R₂ are, independently from each other, selected from the group consisting of H, NO₂, CN, halogen, (C₁-C₄)alkyl; (C₂-C₄)alkenyl; (C₁-C₄)alkoxy(C₁-C₄)alkyl, (C₃-C₇)cycloalkyl(C₁-C₄)alkyl, (C₁-C₄)alkyl substituted by a (5-9)-membered heteroaromatic ring comprising from 1 to 3 heteroatoms selected from O, N, and S, amino optionally substituted with bis(dimethylamino)methylene, pyridyl, (C₁-C₃)alkyl-SO₂—,
a group NHSO₂(C₁-C₃)alkyl;
a group N(SO₂(C₁-C₃)alkyl)₂;
a group NH—CO—R₃,
wherein R₃ is selected from the group consisting of benzyl, (C₃-C₇)cycloalkyl(C₁-C₄)alkyl, phenyl, vinyl, halogen-CH₂—, (C₁-C₃)alkyl;
a group NH—CH₂—R₄
wherein R₄ is an optionally substituted phenyl ring or an optionally substituted (5-9)-membered heteroaromatic ring comprising from 1 to 3 heteroatoms selected from O, N, and S; or
R₁ and R₂ together form an optionally substituted aromatic ring comprising 6 carbon atoms.

In a preferred and advantageous embodiment R₁ or R₂ is a group NH—CH₂—R₄ wherein R₄ is an optionally substituted aromatic ring or an optionally substituted (5-9)-membered heteroaromatic ring comprising from 1 to 3 heteroatoms selected from O, N, and S.

In a further aspect, the invention relates to a tetrahydroquinoline compound of Formula (I) or a pharmaceutically acceptable salt thereof for use as a medicament.

In a still further aspect, the invention relates to a tetrahydroquinoline compound of Formula (I) or a pharmaceutically acceptable salt thereof for use as a disruptor of the FANCM/BTR interaction so as to hamper the FANCM localization to telomeres, preferably in the treatment of tumors.

### BRIEF DESCRIPTION OF THE DRAWINGS

FIG. 1 reports Scheme 1 of the general procedure for the synthesis of compounds 1-6, 14, 19, and 20.

FIG. 2 reports Scheme 2 of the general procedure for the synthesis of compounds 8, 9, 11-13, 16-18, and 21-32.

FIG. 3 reports Scheme 3 of the general procedure for the synthesis of compounds 7, 10, and 15.

FIG. 4 reports in a) Analysis of drug treatments on inhibiting U2OS cell line growth by 50% (left) and on causing 50% of cell death in the same cell line (right). Error bars represented mean±SEM from n=3 experiments. In b) Comparison of IC₅₀ (left) and EC₅₀ (right) values across the panel of cell lines treated with compound 32. Each dot represented IC₅₀ and EC₅₀ values calculated from drugs that successfully inhibited growth or caused cell death in cell lines. The bar represents the mean IC₅₀ and EC₅₀ values within each group. In c) and d) Co-immunoprecipitation using RMI1 antibody to determine if the FANCM-BTR interaction is inhibited by compound 32. U2OS cells were treated with compound 32 (IC₅₀=15.51 μM). Shown is a western blot (g) of co-immunoprecipitation products, with 5% input shown in (top) and eluate in (bottom). Eluates were normalized to and expressed as a percentage of their respective inputs. Quantitation was performed using ImageJ software.

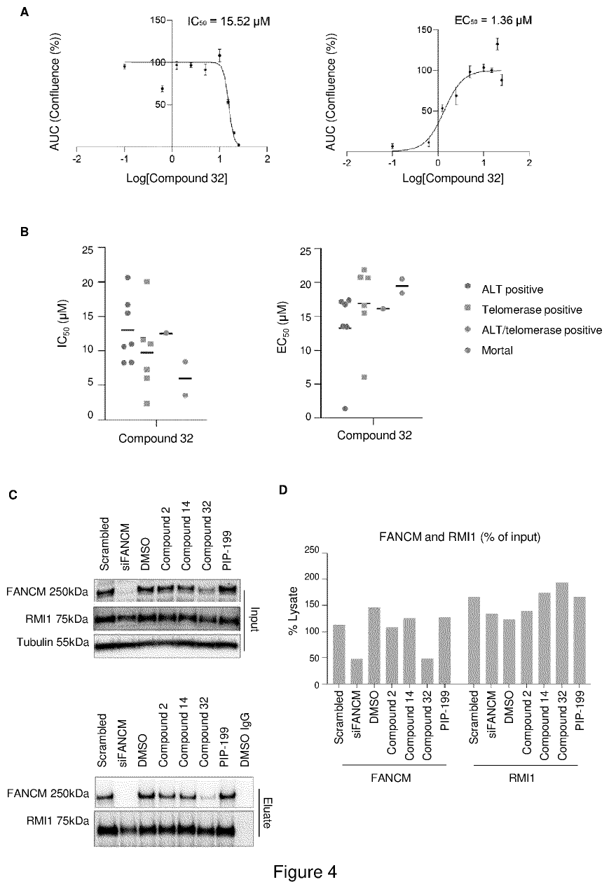

FIG. 5 reports the cell proliferation graphs of different cell lines compounds 1, 2, 14, and 32 treatment. Cells were treated with compounds at concentrations of 0 μM (0.5% DMSO), 0.625 μM, 1.25 μM, 2.5 μM, 5 μM, 10 μM, 15 μM, 20 μM and 25 μM. Cell proliferation was measured by % confluency. Error bars represent mean±SEM from n=3 experiments.

### DETAILED DESCRIPTION OF THE INVENTION

Therefore, the present invention concerns a tetrahydroquinoline compound of Formula (I) or a pharmaceutically acceptable salt thereof

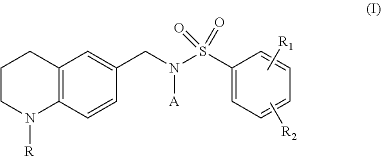

wherein
A is (C₃-C₇)cycloalkyl; (C₃-C₇)heterocycloalkyl comprising from 1 to 3 heteroatoms selected from O, N, and S; (C₁-C₇)alkyl, (C₁-C₇)heteroalkyl comprising from 1 to 3 heteroatoms selected from O, N, and S; an optionally substituted phenyl ring or an optionally substituted (5-9)-membered heteroaromatic ring comprising from 1 to 3 heteroatoms selected from O, N, and S;
R is H, (C₁-C₄)alkyl; (C₁-C₃)alkoxy(C₁-C₃)alkyl, (C₃-C₇)cycloalkyl;
R₁ and R₂ are, independently from each other, selected from the group consisting of H, NO₂, CN, halogen, (C₁-C₄)alkyl; (C₂-C₄)alkenyl; (C₁-C₄)alkoxy(C₁-C₄)alkyl, (C₃-C₇)cycloalkyl(C₁-C₄)alkyl, (C₁-C₄)alkyl substituted by a (5-9)-membered heteroaromatic ring comprising from 1 to 3 heteroatoms selected from O, N, and S, amino optionally substituted with bis(dimethylamino)methylene, pyridyl, trifluoromethyl-SO₂—, (C₁-C₃)alkyl-SO₂—,
a group NH—CO—R₃,
wherein R₃ is selected from the group consisting of benzyl, (C₃-C₇)cycloalkyl(C₁-C₄)alkyl, phenyl, vinyl, halogen-CH₂—, (C₁-C₃)alkyl;
a group NH—CH₂—R₄
wherein R₄ is an optionally substituted phenyl ring or an optionally substituted (5-9)-membered heteroaromatic ring comprising from 1 to 3 heteroatoms selected from O, N, and S; or
R₁ and R₂ together form an optionally substituted aromatic ring comprising 6 carbon atoms.

When in the present invention the following terms are used:
“(C₁-C₄)alkyl” it is meant a linear or branched alkyl containing from 1 to 4 carbon atoms, such as methyl, ethyl, n-propyl, isopropyl, n-butyl, tert-butyl, sec-butyl and iso-butyl and the like;
“(C₂-C₄)alkenyl” it is meant an alkenyl containing from 2 to 4 carbon atoms, such as vinyl, 1-propene; 2-propene, 1-butene, 1,2-butadiene, and the like.
“(C₁-C₃)alkoxy, it is meant a linear or branched alkoxyl containing from 1 to 3 carbon atoms, such as methoxy, ethoxy, n-propoxy, isopropoxy, and the like
(C₃-C₇)cycloalkyl it is meant a 3- to 7-membered all-carbon cyclic ring such as cyclopropyl, cyclobutyl, cyclopentyl, cyclohexyl, cyclohexenyl and cycloeptenyl;
(C₃-C₇)heterocycloalkyl comprising from 1 to 3 heteroatoms selected from O, N, and S it is meant 3- to 7-membered all-carbon cyclic ring wherein from 1 to 3 carbon are substituted with an hetero atom selected from O, N and S, such as oxiran-2-yl, oxiren-2-yl, aziridin-2-yl, 1H-azirin-2-yl, 2H-azirin-2-yl, 2H-azirin-3-yl, thiiran-2-yl, thiiren-2-yl, thietan-2-yl, thietan-2-yl, 2H-thiet-3-yl, 2H-oxet-3-yl, oxetan-3-yl, oxetan-2-yl, 2-pyrrolidinyl, 2-tetrahydrofuranyl, 2-tetrahydrothiophenyl, 2-piperidinyl, 2-tetrahydropyranyl, 2-thianlyl, 2-azepanyl, 2-oxepanyl, 2-thiepanyl and the like;
(C₁-C₇)heteroalkyl comprising from 1 to 3 heteroatoms selected from O, N, and S it is meant a linear or branched alkyl containing from 1 to 7 carbon atoms wherein from 1 to 3 carbon atoms are substituted with an hetero atom selected from O, N and S, such as methoxyl, ethoxyl, propoxyl, butoxyl, pentoxyl, exyloxyl, pentyloxymethyl, butoxymethyl, propoxymethyl, ethoxymethyl, methoxymethyl, methylamino, ethylamino, propylamino, butylamino, pentylamino, methylthio, ethylthio, propylthio, butylthio, pentylthio and the like;
“(5-9)-membered heteroaromatic ring comprising from 1 to 3 heteroatoms selected from O, N and S” it is meant a 5- to 9-membered aromatic carbocyclic ring where one or more carbon atoms are replaced by heteroatoms such as nitrogen, oxygen and sulfur; the heteroaromatic ring can be optionally further fused to aromatic rings. Non limiting examples of (5-9)-membered heteroaromatic ring are, for instance, pyrrol-2-yl, pyrrol-3-yl, furan-2-yl, furan-3-yl, thiophen-2-yl, thiophen-3-yl, imidazol-4-yl, 1,3-oxazol-5-yl, 1,3-oxazol-4-yl, 1,3-thiazol-5-yl, 1,3-thiazol-4-yl, 1,2,3-triazol-4-yl, 1,2,4-triazol-5-yl, pyridin-2-yl, pyridin-3-yl, pyridin-4-yl, pyrimidin-4-yl, pyrimidin-2-yl, pyrimidin-5-yl, pyrazin-2-yl, indol-2-yl, indol-3-yl, isoindol-1-yl, 1-benzofuran-3-yl, 1-benzofuran-2-yl, 2-benzofuran-1-yl, 2-benzothiophen-1-yl, 1-benzothiophen-3-yl, 2-benzothiophen-3-yl, benzimidazol-2-yl.

According to the present invention and unless otherwise provided, when any of the above-mentioned groups are optionally substituted, it may be substituted in any of its free position by one or more linear or branched (C₁-C₄) alkyl groups or by a halogen atom.

The term “pharmaceutically acceptable salt” of compounds of formula (I) refers to those salts that retain the biological effectiveness and properties of the compound, therefore pharmaceutically acceptable salts of the compounds of formula (I) include the acid addition salts with inorganic or organic acids, e.g., nitric, hydrochloric, hydrobromic, sulfuric, perchloric, phosphoric, acetic, trifluoroacetic, propionic, glycolic, (D) or (L) lactic, oxalic, ascorbic, fumaric, malonic, malic, maleic, tartaric, citric, benzoic, cinnamic, mandelic, methanesulfonic, ethanesulfonic, p-toluenesulfonic, isethionic, succinic and salicylic acid.

In a preferred and advantageous embodiment R₁ or R₂ is a group NH—CH₂—R₄ wherein R₄ is an optionally substituted aromatic ring or an optionally substituted (5-9)-membered heteroaromatic ring comprising from 1 to 3 heteroatoms selected from O, N and S.

Without being bound to any theory the inventors deem that the compounds of Formula (I), due to their specific structure are capable to inhibit FANCM activity or expression, such as inhibiting FANCM's interaction with RMI and/or FANCM's ATPase activity, to inhibit the growth and/or proliferation of ALT tumor cells and/or to induce death of ATL tumor cells.

According to the invention, A is (C₃-C₇)cycloalkyl; (C₃-C₇)heterocycloalkyl comprising from 1 to 3 heteroatoms selected from O, N, and S; (C₁-C₇)alkyl, (C₁-C₇)heteroalkyl comprising from 1 to 3 heteroatoms selected from O, N, and S; an optionally substituted phenyl ring or an optionally substituted (5-9)-membered heteroaromatic ring comprising from 1 to 3 heteroatoms selected from O, N, and S.

In a preferred embodiment, A is (C₃-C₇)cycloalkyl, more preferably A is cyclopentyl.

When A is (C₃-C₇)cycloalkyl, it is preferably selected from the group consisting of cyclopropyl, cyclobutyl, cyclopentyl, cyclohexyl and cycloheptyl, more preferably A is cyclopentyl.

When A is (C₃-C₇)heterocycloalkyl comprising from 1 to 3 heteroatoms selected from O, N, and S, it is preferably selected from the group consisting of Pyrrolidin-2-yl, tetrahydrofuran-2-yl, tetrahydrothiophen-2-yl.

When A is (C₁-C₇)alkyl, it is preferably selected from the group consisting of methyl, ethyl, propyl, butyl, pentyl, hexyl, propan-2-yl, butan-2-yl, pentan-2-yl, hexan-2-yl, pentan-3-yl, 2-methylpentan-3-yl, 2,4-dimethylpentan-3-yl, tert-butyl, 2-methylbutyl, 3-methylbutyl, 2-metylpentyl, 3-metylpentyl and 4-metylpentyl.

When A is (C₁-C₇)heteroalkyl comprising from 1 to 3 heteroatoms selected from O, N, and S, it is preferably selected from the group consisting of methoxyl, propoxyl, butoxyl, pentoxyl, hexyloxyl, pentyloxymethyl.

When A is of (5-9)-membered heteroaromatic ring comprising from 1 to 3 heteroatoms selected from O, N and S, it is selected from the group consisting of pyrrolyl, pyrrol-2-yl, pyrrol-3-yl, furan-2-yl, furan-3-yl, thiophen-2-yl, thiophen-3-yl, imidazol-4-yl, 1,3-oxazol-5-yl, 1,3-oxazol-4-yl, 1,3-thiazol-5-yl, 1,3-thiazol-4-yl, 1,2,3-triazol-4-yl, 1,2,4-triazol-5-yl, pyridin-2-yl, pyridin-3-yl, pyridin-4-yl, pyrimidin-4-yl, pyrimidin-2-yl, pyrimidin-5-yl, pyrazin-2-yl, indol-2-yl, indol-3-yl, isoindol-1-yl, 1-benzofuran-3-yl, 1-benzofuran-2-yl, 2-benzofuran-1-yl, 2-benzothiophen-1-yl, 1-benzothiophen-3-yl, 2-benzothiophen-3-yl and benzimidazol-2-yl.

R is H, (C₁-C₄)alkyl; (C₁-C₃)alkoxy(C₁-C₃)alkyl or (C₃-C₇)cycloalkyl.

In a preferred embodiment R is (C₁-C₄)alkyl, more preferably it is ethyl.

When R is (C₁-C₄)alkyl, it is preferably selected from the group consisting of methyl, ethyl, n-propyl, isopropyl, n-butyl, tert-butyl, sec-butyl and iso-butyl, more preferably ethyl.

When R is (C₁-C₃)alkoxy, it is preferably a methoxyl.

When R is (C₁-C₃)alkyl, it is preferably an ethyl.

When R is (C₃-C₇)cycloalkyl, it is preferably a cyclopropyl.

According to the invention R₁ and R₂ are, independently from each other, selected from the group consisting of H, NO₂, CN, halogen, (C₁-C₄)alkyl; (C₂-C₄)alkenyl; (C₁-C₄)alkoxy(c₁-C₄)alkyl, (C₃-C₇)cycloalkyl(C₁-C₄)alkyl, (5-9)-membered heteroaromatic ring comprising from 1 to 3 heteroatoms selected from O, N, and S, amino optionally substituted with bis(dimethylamino)methylene, pyridyl, trifluoromethyl-SO2-, (C₁-C₃)alkyl-SO₂—,
a group NHSO₂(C₁-C₃)alkyl;
a group N(SO₂(C₁-C₃)alkyl)₂;
a group NH—CO—R₃,
wherein R₃ is selected from the group consisting of benzyl, (C₃-C₇)cycloalkyl(C₁-C₄)alkyl, phenyl, vinyl, halogen-CH₂—, (C₁-C₃)alkyl;
a group NH—CH₂—R₄
wherein R₄ is an optionally substituted phenyl ring or an optionally substituted (5-9)-membered heteroaromatic ring comprising from 1 to 3 heteroatoms selected from O, N and S; and
R₁ and R₂ together form an optionally substituted aromatic ring comprising 6 carbon atoms.

In a preferred embodiment one of R₁ or R₂ is H.

In a more preferred embodiment one of R₁ and R₂ is H and the other is a group NH—CH₂—R₄ wherein R₄ is an optionally substituted phenyl ring or an optionally substituted (5-9)-membered heteroaromatic ring comprising from 1 to 3 heteroatoms selected from O, N and S.

In a still more preferred embodiment R₄ is selected from the group consisting of pyrrol-2-yl, pyrrol-3-yl, furan-2-yl, furan-3-yl, thiophen-2-yl, thiophen-3-yl, imidazol-4-yl, 1,3-oxazol-5-yl, 1,3-oxazol-4-yl, 1,3-thiazol-5-yl, 1,3-thiazol-4-yl, 1,2,3-triazol-4-yl, 1,2,4-triazol-5-yl, pyridin-2-yl, pyridin-3-yl, pyridin-4-yl, pyrimidin-4-yl, pyrimidin-2-yl, pyrimidin-5-yl, pyrazin-2-yl, indol-2-yl, indol-3-yl, isoindol-1-yl, 1-benzofuran-3-yl, 1-benzofuran-2-yl, 2-benzofuran-1-yl, 2-benzothiophen-1-yl, 1-benzothiophen-3-yl, 2-benzothiophen-3-yl, benzimidazol-2-yl. R₄ is in a still more preferred and advantageous embodiment imidazol-4-yl.

R₄ can be an optionally substituted phenyl ring. Preferably it is phenyl substituted with halogen, more preferably with bromine.

When R₁ or R₂ is halogen, it is preferably bromine.

When R₁ or R₂ is (C₁-C₄)alkyl, it is preferably selected from butan-2-yl (or sec-butyl) or propan-2-yl (or isobutyl).

When R₁ or R₂ is (C₂-C₄)alkenyl, it is preferably vinyl.

When R₁ or R₂ is (C₁-C₄)alkoxy(C₁-C₄)alkyl, it is preferably methoxyethyl.

When R₁ or R₂ is (C₁-C₄)alkyl substituted by a (5-9)-membered heteroaromatic ring comprising from 1 to 3 heteroatoms selected from O, N, and S, it is preferably furan-2-ylmethyl.

When R₁ or R₂ is (C₃-C₇)cycloalkyl(C₁-C₄)alkyl, it is preferably 3-(cyclopentyl)methyl.

When R₁ or R₂ is amino optionally substituted with bis(dimethylamino)methylene, pyridyl, trifluoromethyl-SO₂—, (C₁-C₃)alkyl-SO₂—, it is preferably selected from amino substituted with bis(dimethylamino)methylene, pyridyl, trifluoromethyl-SO₂—, methyl-SO₂—.

R₁ or R₂ can be a group NH—CO—R₃, wherein R₃ is selected from the group consisting of benzyl, (C₃-C₇)cycloalkyl(C₁-C₄)alkyl, phenyl, vinyl, halogen-CH₂—, (C₁-C₃)alkyl.

Preferably R₃ is selected from the group consisting of benzyl, cyclopentylethyl, phenyl, vinyl, bromo-CH₂—, and ethyl.

R₁ and R₂ together can also form an optionally substituted aromatic ring comprising 6 carbon atoms.

In a preferred embodiment, the compound of formula (I) is selected from the group consisting of

| CODE | STRUCTURE | IUPAC Name |
|---|---|---|
| Compound 1 | 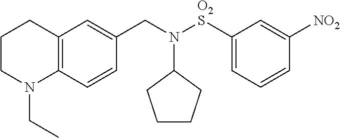 [SMILES: CCN1CCCc2cc(CN(C3CCCC3)S(=O)(=O)c3cccc([N+](=O)[O-])c3)ccc21] | N-cyclopentyl-N-((1-ethyl- 1,2,3,4-tetrahydroquinolin-6- yl)methyl)-3- nitrobenzenesulfonamide |
| Compound 2 | 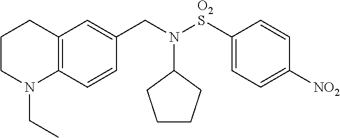 [SMILES: CCN1CCCc2cc(CN(C3CCCC3)S(=O)(=O)c3ccc([N+](=O)[O-])cc3)ccc21] | N-cyclopentyl-N-((1-ethyl- yl)methyl)-4- 1,2,3,4-tetrahydroquinolin-6- nitrobenzenesulfonamide |
| Compound 3 | 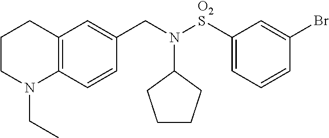 [SMILES: CCN1CCCc2cc(CN(C3CCCC3)S(=O)(=O)c3cccc(Br)c3)ccc21] | 3-bromo-N-cyclopentyl-N- ((1-ethyl-1,2,3,4- tetrahydroquinolin-6- yl)methyl) benzenesulfonamide |
| Compound 4 | 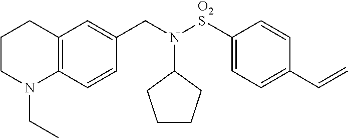 [SMILES: C=Cc1ccc(S(=O)(=O)N(Cc2ccc3c(c2)CCCN3CC)C2CCCC2)cc1] | N-cyclopentyl-N-((1-ethyl- 1,2,3,4-tetrahydroquinolin-6- yl)methyl)-4- vinylbenzenesulfonamide |
| Compound 5 | 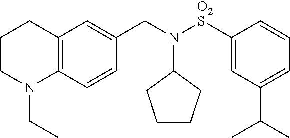 [SMILES: CCN1CCCc2cc(CN(C3CCCC3)S(=O)(=O)c3cccc(C(C)C)c3)ccc21] | N-cyclopentyl-N-((1-ethyl- 1,2,3,4-tetrahydroquinolin-6- yl)methyl)-3- isopropylbenzenesulfonamide |
| Compound 6 | 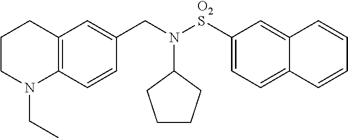 [SMILES: CCN1CCCc2cc(CN(C3CCCC3)S(=O)(=O)c3ccc4ccccc4c3)ccc21] | N-cyclopentyl-N-((1-ethyl- 1,2,3,4-tetrahydroquinolin-6- yl)methyl)naphthalene-2- sulfonamide |
| Compound 7 | 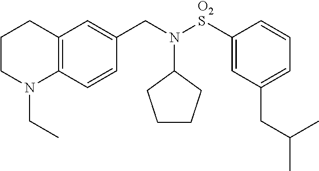 [SMILES: CCN1CCCc2cc(CN(C3CCCC3)S(=O)(=O)c3cccc(CC(C)C)c3)ccc21] | N-cyclopentyl-N-((1-ethyl- 1,2,3,4-tetrahydroquinolin-6- yl)methyl)-3- isobutylbenzenesulfonamide |
| Compound 8 | 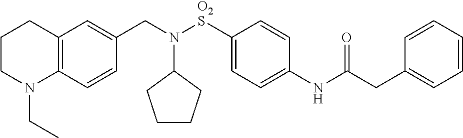 [SMILES: CCN1CCCc2cc(CN(C3CCCC3)S(=O)(=O)c3ccc(NC(=O)Cc4ccccc4)cc3)ccc21] | N-(4-(N-cyclopentyl-N-((1- ethyl-1,2,3,4- tetrahydroquinolin-6- yl)methyl)sulfamoyl)phenyl)- 2-phenylacetamide |
| Compound 9 | 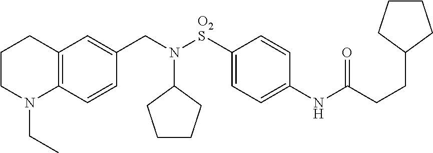 [SMILES: CCN1CCCc2cc(CN(C3CCCC3)S(=O)(=O)c3ccc(NC(=O)CCC4CCCC4)cc3)ccc21] | 3-cyclopentyl-N-(4-(N- cyclopentyl-N-((1-ethyl- 1,2,3,4-tetrahydroquinolin-6- l)methyl)sulfamoyl)phenyl) propanamide |
| Compound 10 | 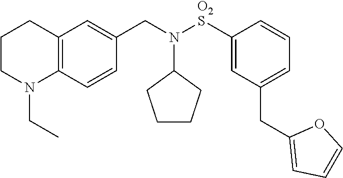 [SMILES: CCN1CCCc2cc(CN(C3CCCC3)S(=O)(=O)c3cccc(Cc4ccco4)c3)ccc21] | N-cyclopentyl-N-((1-ethyl- 1,2,3,4-tetrahydroquinolin-6- yl)methyl)-3-(furan-2- ylmethyl) benzenesulfonamide |
| Compound 11 | 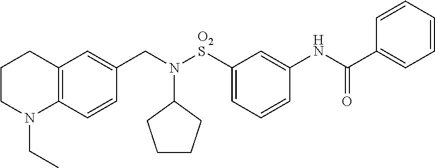 [SMILES: CCN1CCCc2cc(CN(C3CCCC3)S(=O)(=O)c3cccc(NC(=O)c4ccccc4)c3)ccc21] | N-(3-(N-cyclopentyl-N-((1- ethyl-1,2,3,4- tetrahydroquinolin-6- yl)methyl)sulfamoyl)phenyl) benzamide |
| Compound 12 | 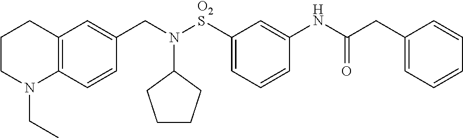 [SMILES: CCN1CCCc2cc(CN(C3CCCC3)S(=O)(=O)c3cccc(NC(=O)Cc4ccccc4)c3)ccc21] | N-(3-(N-cyclopentyl-N-((1- ethyl-1,2,3,4- tetrahydroquinolin-6- yl)methyl)sulfamoyl)phenyl)- 2-phenylacetamide |
| Compound 13 | 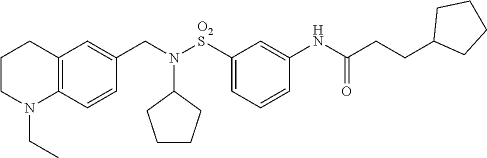 [SMILES: CCN1CCCc2cc(CN(C3CCCC3)S(=O)(=O)c3cccc(NC(=O)CCC4CCCC4)c3)ccc21] | 3-cyclopentyl-N-(3-(N- cyclopentyl-N-((1-ethyl- 1,2,3,4-tetrahydroquinolin-6- yl)methyl)sulfamoyl)phenyl) propanamide |
| Compound 14 | 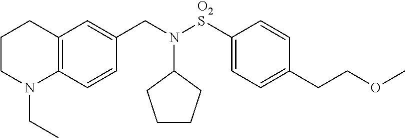 [SMILES: CCN1CCCc2cc(CN(C3CCCC3)S(=O)(=O)c3ccc(CCOC)cc3)ccc21] | N-cyclopentyl-N-((1-ethyl- 1,2,3,4-tetrahydroquinolin-6- yl)methyl)-4-(2- methoxyethyl) benzenesulfonamide |
| Compound 15 | 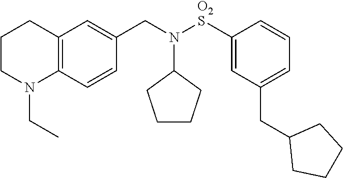 [SMILES: CCN1CCCc2cc(CN(C3CCCC3)S(=O)(=O)c3cccc(CC4CCCC4)c3)ccc21] | N-cyclopentyl-3- (cyclopentylmethyl)-N-((1- ethyl-1,2,3,4- tetrahydroquinolin-6- yl)methyl) benzenesulfonamide |
| Compound 16 | 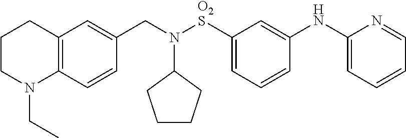 [SMILES: CCN1CCCc2cc(CN(C3CCCC3)S(=O)(=O)c3cccc(Nc4ccccn4)c3)ccc21] | N-cyclopentyl-N-((1-ethyl- 1,2,3,4-tetrahydroquinolin-6- yl)methyl)-3-(pyridin-2- ylamino) benzenesulfonamide |
| Compound 17 | 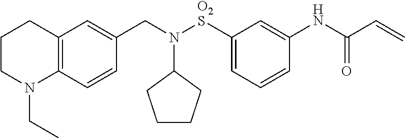 [SMILES: C=CC(=O)Nc1cccc(S(=O)(=O)N(Cc2ccc3c(c2)CCCN3CC)C2CCCC2)c1] | N-(3-(N-cyclopentyl-N-((1- ethyl-1,2,3,4- tetrahydroquinolin-6- yl)methyl)sulfamoyl)phenyl) acrylamide |
| Compound 18 | 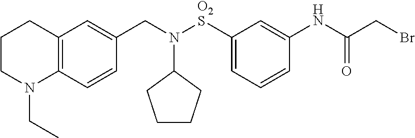 [SMILES: CCN1CCCc2cc(CN(C3CCCC3)S(=O)(=O)c3cccc(NC(=O)CBr)c3)ccc21] | 2-bromo-N-(3-(N- cyclopentyl-N-((1-ethyl- 1,2,3,4-tetrahydroquinolin-6- yl)methyl)sulfamoyl) phenyl)acetamide |
| Compound 19 | 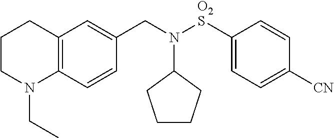 [SMILES: CCN1CCCc2cc(CN(C3CCCC3)S(=O)(=O)c3ccc(C#N)cc3)ccc21] | 4-cyano-N-cyclopentyl-N- ((1-ethyl-1,2,3,4- tetrahydroquinolin-6- yl)methyl) benzenesulfonamide |
| Compound 20 | 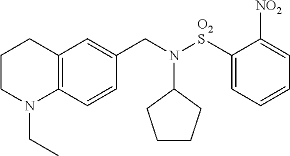 [SMILES: CCN1CCCc2cc(CN(C3CCCC3)S(=O)(=O)c3ccccc3[N+](=O)[O-])ccc21] | N-cyclopentyl-N-((1-ethyl- 1,2,3,4-tetrahydroquinolin-6- yl)methyl)-2- nitrobenzenesulfonamide |
| Compound 21 | 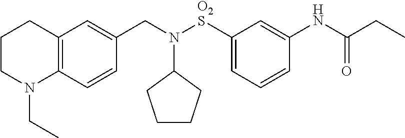 [SMILES: CCC(=O)Nc1cccc(S(=O)(=O)N(Cc2ccc3c(c2)CCCN3CC)C2CCCC2)c1] | N-(3-(N-cyclopentyl-N-((1- ethyl-1,2,3,4- tetrahydroquinolin-6- yl)methyl)sulfamoyl)phenyl) propionamide |
| Compound 22 | 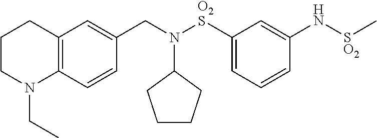 [SMILES: CCN1CCCc2cc(CN(C3CCCC3)S(=O)(=O)c3cccc(NS(C)(=O)=O)c3)ccc21] | N-cyclopentyl-N-((1-ethyl- 1,2,3,4-tetrahydroquinolin-6- yl)methyl)-3- (methylsulfonamido) benzenesulfonamide |
| Compound 23 | 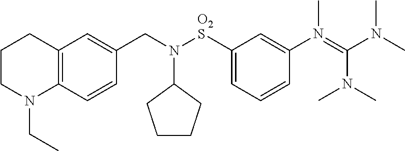 | 3- ((bis(dimethylamino) methylene)amino)-N- cyclopentyl-N- ((1-ethyl-1,2,3,4- tetrahydroquinolin-6- yl)methyl) benzenesulfonamide |
| Compound 24 | 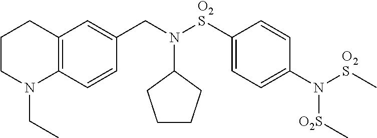 [SMILES: CCN1CCCc2cc(CN(C3CCCC3)S(=O)(=O)c3ccc(N(S(C)(=O)=O)S(C)(=O)=O)cc3)ccc21] | N-cyclopentyl-N-((1-ethyl- 1,2,3,4-tetrahydroquinolin-6- yl)methyl)-4-(N- (methylsulfonyl) methylsulfonamido) benzenesulfonamide |
| Compound 25 | 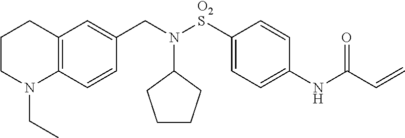 [SMILES: C=CC(=O)Nc1ccc(S(=O)(=O)N(Cc2ccc3c(c2)CCCN3CC)C2CCCC2)cc1] | N-(4-(N-cyclopentyl-N-((1- ethyl-1,2,3,4- tetrahydroquinolin-6- yl)methyl)sulfamoyl) phenyl)acrylamide |
| Compound 26 | 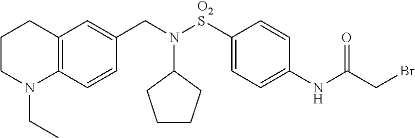 [SMILES: CCN1CCCc2cc(CN(C3CCCC3)S(=O)(=O)c3ccc(NC(=O)CBr)cc3)ccc21] | 2-bromo-N-(4-(N- cyclopentyl-N-((1-ethyl- 1,2,3,4-tetrahydroquinolin-6- yl)methyl)sulfamoyl) phenyl)acetamide |
| Compound 27 | 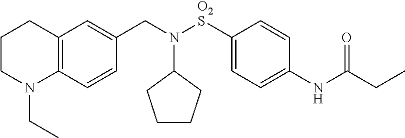 [SMILES: CCC(=O)Nc1ccc(S(=O)(=O)N(Cc2ccc3c(c2)CCCN3CC)C2CCCC2)cc1] | N-(4-(N-cyclopentyl-N-((1- ethyl-1,2,3,4- tetrahydroquinolin-6- yl)methyl)sulfamoyl) phenyl)propionamide |
| Compound 28 | 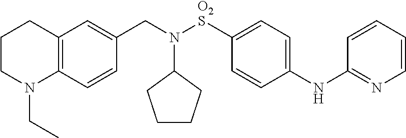 [SMILES: CCN1CCCc2cc(CN(C3CCCC3)S(=O)(=O)c3ccc(Nc4ccccn4)cc3)ccc21] | N-cyclopentyl-N-((1-ethyl- 1,2,3,4-tetrahydroquinolin-6- yl)methyl)-4-(pyridin-2- ylamino) benzenesulfonamide |
| Compound 29 | 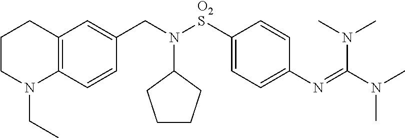 [SMILES: CCN1CCCc2cc(CN(C3CCCC3)S(=O)(=O)c3ccc(N=C(N(C)C)N(C)C)cc3)ccc21] | 4- ((bis(dimethylamino) methylene)amino)-N- cyclopentyl-N- ((1-ethyl-1,2,3,4- tetrahydroquinolin-6- yl)methyl) benzenesulfonamide |
| Compound 30 | 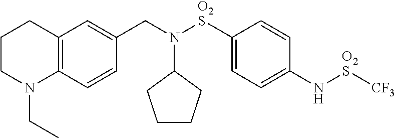 [SMILES: CCN1CCCc2cc(CN(C3CCCC3)S(=O)(=O)c3ccc(NS(=O)(=O)C(F)(F)F)cc3)ccc21] | N-cyclopentyl-N-((1-ethyl- 1,2,3,4-tetrahydroquinolin- 6-yl)methyl)-4- (trifluoromethylsulfonamido) benzenesulfonamide |
| Compound 31 | 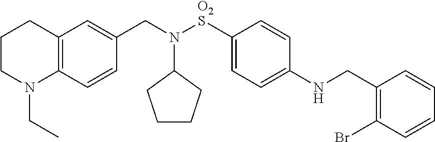 [SMILES: CCN1CCCc2cc(CN(C3CCCC3)S(=O)(=O)c3ccc(NCc4ccccc4Br)cc3)ccc21] | 4-((2-bromobenzyl)amino)- N-cyclopentyl-N-((1-ethyl- 1,2,3,4-tetrahydroquinolin-6- yl)methyl) benzenesulfonamide |
| Compound 32 | 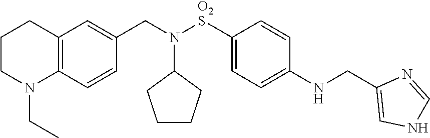 [SMILES: CCN1CCCc2cc(CN(C3CCCC3)S(=O)(=O)c3ccc(NCc4c[nH]cn4)cc3)ccc21] | 4-(((1H-imidazol-4- yl)methyl)amino)-N- cyclopentyl-N-((1-ethyl- 1,2,3,4-tetrahydroquinolin- 6-yl)methyl) benzenesulfonamide |

Preferably the tetrahydroquinoline compound of Formula (I) or a pharmaceutically acceptable salt thereof is compound 32, 1, 2 and 14. More preferably it is compound 32.

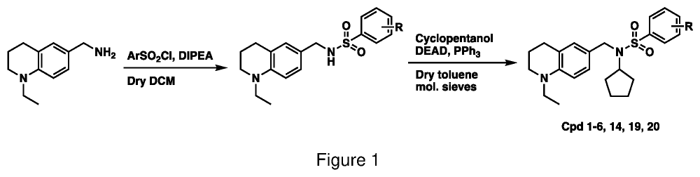

Compounds 1-6, 14, 19, and 20 were prepared as reported in Scheme 1 (FIG. 1).

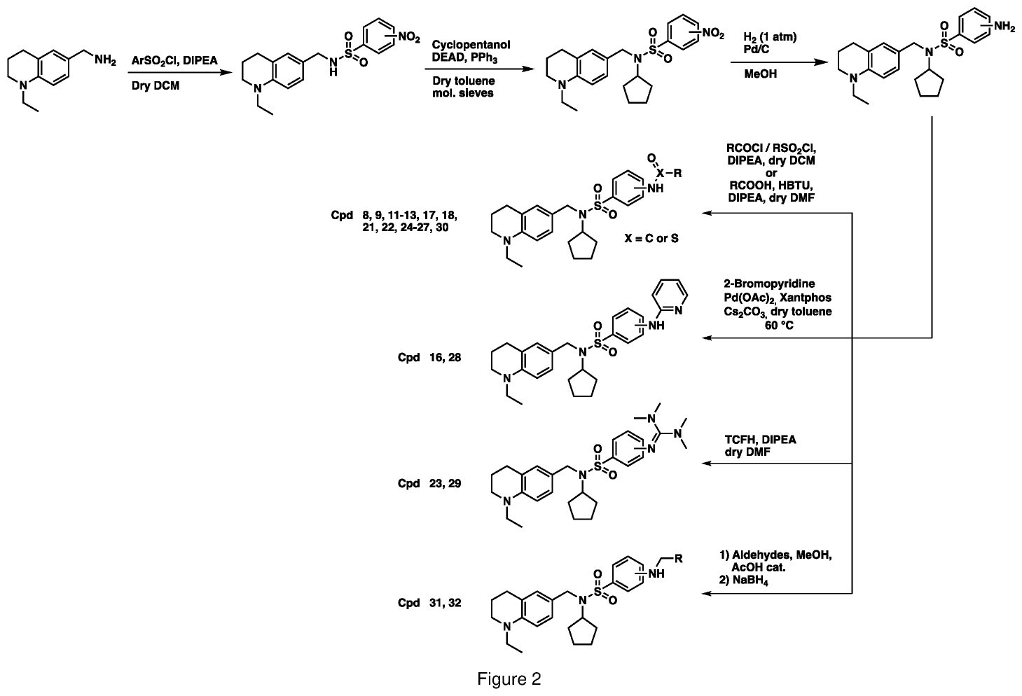

Compounds 8, 9, 11-13, 16-18, and 21-32 were obtained by following the general procedures reported in Scheme 2 (FIG. 2).

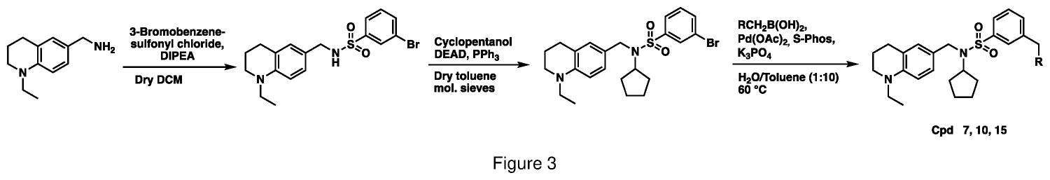

Compounds 7, 10, and 15 were obtained following the route reported in Scheme 3 (FIG. 3).

Preferably the compounds of the invention are present in a pharmaceutical composition together with pharmaceutically acceptable carriers and excipients.

In a further aspect hence, the invention concerns the composition of the invention and at least one pharmaceutically acceptable excipient.

The composition hence can comprise also pharmaceutically acceptable excipients and can be administered in a pharmaceutical form suitable for the desired administration route.

Pharmaceutically acceptable additives can be excipients, ligands, dispersing agents, colorants, and humectants, commonly used for the preparation of tablets, capsules, pills, solutions, suspensions, and emulsions for oral administration.

Injectable solutions are also contemplated for parental administration, comprising subcutaneous, spinal, and transdermal administration.

The compound of Formula (I) can be used as a free base or in a salt form.

Preferably, the salt is a salt selected from the group consisting of hydrochloride, hydrobromide, phosphate, sulfate, hydrogensulphate, alkylsulphonate, arylsulphonate, acetate, citrate, oxalate, maleate, fumarate, succinate, lactate, and tartrate. Salt can also be formed between a cation and a negatively charged group. Suitable cations include potassium ion, magnesium ion, calcium ion, and an ammonium cation such as tetramethylammonium ion. Examples of some suitably substituted ammonium ions are those derived from ethylamine, diethylamine, triethylamine, ethanolamine, diethanolamine, piperazine, choline, meglumine, and tromethamine, as well as amino acids, such as lysine and arginine.

The pharmaceutical composition according to the present invention is preferably for intra-articular, intravenous, oral, transdermal, intrathecal, intranasal, intraperitoneal or intramuscular administration, more preferably oral administration.

The compound of Formula (I) of the invention is preferably in a dose in the range from 1 nM to 20000 nM, more preferably in a dose in the range from 1 nM to 1000 nM.

In a further aspect the invention relates to a tetrahydroquinoline compound of Formula (I) or a pharmaceutically acceptable salt thereof for use as a medicament.

In a still further aspect, the invention relates to a tetrahydroquinoline compound of Formula (I) or a pharmaceutically acceptable salt thereof for use as a disruptor of the FANCM/BTR interaction so as to hamper the FANCM localization to telomeres, preferably in the treatment of tumors such as Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Angiosarcoma, Appendix Cancer, Astrocytomas, Atypical Teratoid/Rhabdoid Tumor, Basal Cell Carcinoma of the Skin, Bile Duct Cancer, Bladder Cancer, Brain Tumors, Breast Cancer, Bronchial Tumors, Burkitt Lymphoma, Carcinoid Tumor (Gastrointestinal), Carcinoma of Unknown Primary, Cardiac (Heart) Tumors, Central Nervous System Tumors, Atypical Teratoid/Rhabdoid Tumor, Medulloblastoma and Other CNS Embryonal Tumors, Germ Cell Tumor, Primary CNS Lymphoma, Cervical Cancer, Childhood Cancers, Cholangiocarcinoma, Chondrosarcoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Embryonal Tumors, Medulloblastoma and Other Central Nervous System Cancers, Endometrial Cancer, Ependymoma, Childhood, Esophageal Cancer, Esthesioneuroblastoma, Ewing Sarcoma, Extracranial Germ Cell Tumor, Extragonadal Germ Cell Tumor, Eye Cancer, Intraocular Melanoma, Retinoblastoma, Fallopian Tube Cancer, Gallbladder Cancer, Gastric Cancer, Gastrointestinal Carcinoid Tumor, Gastrointestinal Stromal Tumors (GIST), Germ Cell Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Childhood, Hepatocellular Cancer, Histiocytosis, Langerhans Cell, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leiomyoma, Leukemia, Lip and Oral Cavity Cancer, Liposarcoma, Liver Cancer, Lung Cancer (including Non-Small Cell, Small Cell, Pleuropulmonary Blastoma, Pulmonary Inflammatory Myofibroblastic Tumor, and Tracheobronchial Tumor), Leiomyosarcoma, Lymphoma, Male Breast Cancer, Malignant Fibrous Histiocytoma, Melanoma, Melanoma, Intraocular (Eye), Merkel Cell Carcinoma (Skin Cancer), Mesothelioma, Malignant, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloproliferative Neoplasms, Chronic, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma, Osteoclastoma, Ovarian Cancer, Pancreatic Cancer, Pancreatic Neuroendocrine Tumors (PanNET), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Pulmonary Inflammatory Myofibroblastic Tumor, Rare Cancers of Childhood, Rectal Cancer, Recurrent Cancer, Renal Cell Cancer, Retinoblastoma, Rhabdomyosarcoma, Childhood, Salivary Gland Cancer, Sarcoma, Childhood Rhabdomyosarcoma, Childhood Vascular Tumors, Ewing Sarcoma, Kaposi Sarcoma, Osteosarcoma, Soft Tissue Sarcoma, Uterine Sarcoma, Sézary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Metastatic, Stomach Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Tracheobronchial Tumors, Transitional Cell Cancer of the Renal Pelvis and Ureter, Carcinoma of Unknown Primary, Undifferentiated Pleomorphic Sarcoma, Ureter and Renal Pelvis, Transitional Cell Cancer, Urethral Cancer, Uterine Cancer, Endometrial, Uterine Sarcoma, Vaginal Cancer, Vascular Tumors, Vulvar Cancer, Wilms Tumor and Other Childhood Kidney Tumors.

The inventors found out that the compounds of Formula(I), due to their specific structure are capable to inhibit FANCM activity or expression, such as inhibiting FANCM's interaction with RMI and/or FANCM's ATPase activity, to inhibit the growth and/or proliferation of ALT tumor cells and/or to induce death of ATL tumor cells such as Chondrosarcoma, Osteoclastoma, Osteosarcoma, Breast Cancer, CNS Cancers, Neuroblastoma, Pancreatic Neuroendocrine Tumors (PanNET), Angiosarcoma, Leiomyoma, Leiomyosarcoma, Liposarcoma, Undifferentiated Pleomorphic Sarcoma.

Moreover the invention relates to a tetrahydroquinoline compound of Formula (I) or a pharmaceutically acceptable salt thereof for use as a disruptor of the FANCM/BTR interaction so as to hamper the FANCM localization to telomeres, in the treatment of human disorders such as the Bloom syndrome, Dyskeratosis Congenita, and possibly age-related diseases.

The invention will be now detailed with reference to the preparative examples of the compounds of the invention and examples for testing the antitumor activity with illustrative and not limitative purposes.

### EXPERIMENTAL PART

Compounds of the invention were prepared according to synthetic protocols reported below.

### Materials and Methods

Final compounds were analyzed by analytical HPLC (Shimadzu Prominance HPLC system) equipped with a C18-bounded analytical RP-HPLC column (Phenomenex Kinetex, 4.6 mm×150 mm, 5 μm) using a gradient elution (from 10% to 90% of acetonitrile in water (0.1% TFA) over 20 min; flow rate=1.0 mL/min; diode array UV detector). Molecular weights of compounds were confirmed by ESI-mass spectrometry (MS) using a Q Exactive Orbitrap LC-MS/MS system (Thermo Fisher Scientific, Waltham, MA, USA). 1H NMR and 13C NMR spectra were recorded on a Varian INOVA 500 MHz spectrometer and measured CDCl3 (7.26/77.16 ppm) or DMSO-d₆ (2.50/39.52 ppm). Multiplicities are abbreviated as follows: s=singlet, d=doublet, t=triplet, q=quadruplet, p=pentuplet, hept=heptuplet, dd=doublet of doublet, td=triplet of doublet, m=multiplet, and bs=broad signal. Coupling constant J values are measured in Hertz (Hz), and chemical shift values are in parts per million (ppm).

### Example 1

### A) Preparation of N-((1-ethyl-1,2,3,4-tetrahydroquinolin-6-yl)methyl)-3-nitrobenzenesulfonamide

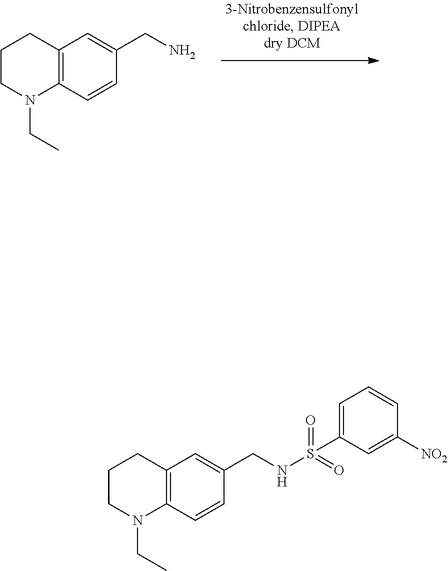 [SMILES: CCN1CCCc2cc(CN)ccc21 ; CCN1CCCc2cc(CNS(=O)(=O)c3cccc([N+](=O)[O-])c3)ccc21]

A stirring solution of (1-ethyl-1,2,3,4-tetrahydroquinolin-6-yl)methanamine (570 mg, 3 mmol, 1 eq) and DIPEA (548 μL, 3.15 mmol, 1.05 eq) in anhydrous DCM (9 mL) was cooled down to 0° C. in an ice bath, then a solution of 3-nitrobenzenesulfonyl chloride (1183 mg, 3.15 mmol, 1.05 eq) in anhydrous DCM (2 mL) was added dropwise and the resulting mixture was allowed to stir overnight at room temperature. The organic solvent was evaporated under reduced pressure, the reaction was extracted in EtOAc and washed with HCl 0.1 M (2×30 mL) and NaHCO₃ sat. sol. (2×50 mL). Each aqueous aliquot was counter extracted with 2 aliquots of EtOAc. The organic phases were collected, dried over anhydrous sodium sulfate, filtered and volatiles evaporated under vacuum to obtain the desired product was obtained as a pale-yellow powder (1010 mg, 92%) which was used in the next steps without any further purification.

¹H NMR (400 MHz, CDCl₃) δ 8.52 (d, J=2.0 Hz, 1H), 8.37-8.31 (m, 1H), 8.11 (dq, J=7.8, 1.2 Hz, 1H), 7.65 (t, J=8.0 Hz, 1H), 6.74 (dd, J=8.3, 2.2 Hz, 1H), 6.63 (d, J=2.2 Hz, 1H), 6.35 (d, J=8.4 Hz, 1H), 4.93 (s, 1H), 4.08 (d, J=5.9 Hz, 2H), 3.26 (q, J=7.0 Hz, 2H), 3.20 (t, J=5.7 Hz, 2H), 2.57 (t, J=6.4 Hz, 2H), 1.86 (p, J=6.1 Hz, 2H), 1.07 (t, J=7.0 Hz, 3H).

¹³C NMR (101 MHz, CDCl₃) δ 148.15, 144.90, 143.07, 132.71, 130.20, 129.28, 127.31, 126.72, 122.57, 121.40, 110.28, 110.14, 48.37, 47.47, 45.35, 28.10, 22.12, 10.87.

According to the above-mentioned protocol the following intermediates were prepared:

### N-((1-Ethyl-1,2,3,4-tetrahydroquinolin-6-yl)methyl)-4-vinylbenzenesulfonamide

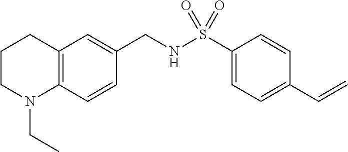 [SMILES: C=Cc1ccc(S(=O)(=O)NCc2ccc3c(c2)CCCN3CC)cc1]

¹H NMR (400 MHz, CDCl₃) δ 7.80 (d, J=8.4 Hz, 2H), 7.51 (d, J=8.3 Hz, 2H), 6.83-6.73 (m, 2H), 6.68 (d, J=2.2 Hz, 1H), 6.45 (d, J=8.4 Hz, 1H), 5.88 (d, J=17.6 Hz, 1H), 5.43 (d, J=10.9 Hz, 1H), 4.53 (t, J=5.9 Hz, 1H), 3.97 (d, J=5.9 Hz, 3H), 3.29 (q, J=7.1 Hz, 2H), 3.21 (t, J=5.7 Hz, 2H), 2.62 (t, J=6.4 Hz, 2H), 1.96-1.87 (m, 2H), 1.09 (t, J=7.0 Hz, 3H).

¹³C NMR (101 MHz, CDCl₃) δ 144.87, 141.77, 139.08, 135.57, 129.27, 127.65, 127.15, 126.78, 122.72, 122.34, 117.33, 110.47, 48.44, 47.27, 45.40, 28.15, 22.22, 10.81.

### N-((1-Ethyl-1,2,3,4-tetrahydroquinolin-6-yl)methyl)-3-isopropylbenzenesulfonamide

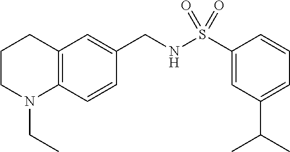 [SMILES: CCN1CCCc2cc(CNS(=O)(=O)c3cccc(C(C)C)c3)ccc21]

¹H NMR (400 MHz, CDCl₃) δ 7.73 (s, 1H), 7.69 (dd, J=6.5, 2.2 Hz, 1H), 7.43 (s, 2H), 6.81 (dd, J=8.4, 2.2 Hz, 1H), 6.71 (d, J=2.2 Hz, 1H), 6.49 (d, J=8.4 Hz, 1H), 4.86 (t, J=5.9 Hz, 1H), 3.98 (d, J=5.9 Hz, 2H), 3.30 (q, J=7.1 Hz, 2H), 3.23 (t, J=5.6 Hz, 2H), 2.97 (hept, J=7.0 Hz, 1H), 2.63 (t, J=6.4 Hz, 2H), 1.90 (p, J=6.1 Hz, 2H), 1.27 (d, J=6.9 Hz, 6H), 1.10 (t, J=7.0 Hz, 3H).

¹³C NMR (101 MHz, CDCl₃) δ 150.19, 140.07, 133.72, 130.77, 129.74, 129.17, 129.04, 127.02, 125.07, 124.68, 118.25, 113.54, 48.30, 47.07, 45.70, 34.12, 27.90, 23.83, 21.87, 10.70.

### N-((1-Ethyl-1,2,3,4-tetrahydroquinolin-6-yl)methyl)naphthalene-2-sulfonamide

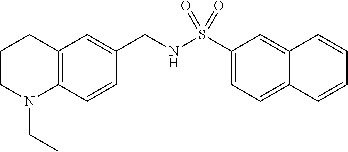 [SMILES: CCN1CCCc2cc(CNS(=O)(=O)c3ccc4ccccc4c3)ccc21]

¹H NMR (400 MHz, CDCl₃) δ 8.39 (d, J=1.8 Hz, 1H), 7.93-7.85 (m, 3H), 7.82 (dd, J=8.6, 1.9 Hz, 1H), 7.65-7.54 (m, 2H), 6.84 (dd, J=8.4, 2.2 Hz, 1H), 6.68 (d, J=2.2 Hz, 1H), 6.48 (d, J=8.4 Hz, 1H), 5.29 (s, 1H), 4.03 (d, J=5.9 Hz, 2H), 3.21 (q, J=7.1 Hz, 2H), 3.15 (t, J=5.7 Hz, 2H), 2.47 (t, J=6.4 Hz, 2H), 1.79 (p, J=6.2 Hz, 2H), 1.07 (t, J=7.0 Hz, 3H).

¹³C NMR (101 MHz, CDCl₃) δ 137.14, 134.69, 132.12, 130.45, 130.32, 129.92, 129.31, 129.28, 128.96, 128.63, 128.48, 128.19, 127.87, 127.40, 127.11, 122.52, 48.14, 47.10, 46.10, 27.50, 21.38, 10.65.

### N-((1-ethyl-1,2,3,4-tetrahydroquinolin-6-yl)methyl)-4-(2-methoxyethyl)benzene-sulfonamide

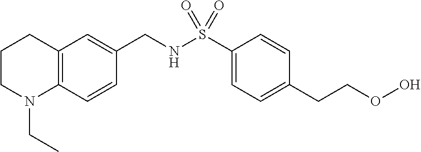 [SMILES: CCN1CCCc2cc(CNS(=O)(=O)c3ccc(CCOO)cc3)ccc21]

¹H NMR (400 MHz, CDCl₃) δ 7.78 (d, J=8.2 Hz, 2H), 7.35 (d, J=8.3 Hz, 2H), 6.81 (dd, J=8.4, 2.3 Hz, 1H), 6.71 (d, J=2.2 Hz, 1H), 6.45 (d, J=8.4 Hz, 1H), 4.77 (t, J=5.8 Hz, 1H), 3.95 (d, J=5.8 Hz, 2H), 3.63 (t, J=6.7 Hz, 2H), 3.35 (s, 3H), 3.29 (q, J=7.1 Hz, 2H), 3.21 (t, J=5.7 Hz, 2H), 2.94 (t, J=6.7 Hz, 2H), 2.63 (t, J=6.4 Hz, 2H), 1.97-1.83 (m, 2H), 1.09 (t, J=7.0 Hz, 3H).

¹³C NMR (101 MHz, CDCl₃) δ 144.69, 144.38, 137.97, 129.49, 129.15, 127.27, 127.02, 122.58, 122.49, 110.38, 72.77, 58.74, 48.33, 47.08, 45.29, 36.03, 28.06, 22.15, 10.71.

### 4-Cyano-N-((1-ethyl-1,2,3,4-tetrahydroquinolin-6-yl)methyl)benzenesulfonamide

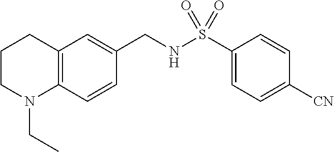 [SMILES: CCN1CCCc2cc(CNS(=O)(=O)c3ccc(C#N)cc3)ccc21]

¹H NMR (400 MHz, CDCl₃) δ 7.86 (d, J=8.0 Hz, 2H), 7.69 (d, J=8.0 Hz, 2H), 6.76 (d, J=7.6 Hz, 1H), 6.61 (s, 1H), 6.39 (d, J=7.6 Hz, 1H), 5.15 (t, J=5.8 Hz, 1H), 4.01 (d, J=5.8 Hz, 2H), 3.29 (q, J=7.1 Hz, 2H), 3.22 (t, J=5.7 Hz, 2H), 2.57 (t, J=6.4 Hz, 2H), 1.88 (p, J=6.1 Hz, 2H), 1.10 (t, J=5.7 Hz, 3H).

¹³C NMR (101 MHz, CDCl₃) δ 144.87, 144.79, 132.67, 129.22, 127.77, 127.21, 122.44, 121.73, 117.55, 115.80, 110.25, 48.31, 47.24, 45.29, 28.06, 22.08, 10.73.

### N-((1-Ethyl-1,2,3,4-tetrahydroquinolin-6-yl)methyl)-2-nitrobenzenesulfonamide

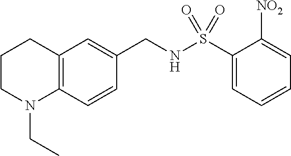 [SMILES: CCN1CCCc2cc(CNS(=O)(=O)c3ccccc3[N+](=O)[O-])ccc21]

¹H NMR (400 MHz, CDCl₃) δ 7.99 (dd, J=7.2, 1.9 Hz, 1H), 7.80 (d, J=7.4 Hz, 1H), 7.70-7.57 (m, 2H), 6.83 (dd, J=8.4, 2.2 Hz, 1H), 6.73 (s, 1H), 6.39 (d, J=8.3 Hz, 1H), 5.19 (s, 1H), 4.14 (d, J=5.8 Hz, 2H), 3.28 (q, J=7.1 Hz, 2H), 3.20 (t, J=5.7 Hz, 2H), 2.59 (t, J=6.5 Hz, 2H), 1.87 (p, J=6.1 Hz, 2H), 1.08 (t, J=7.1 Hz, 3H).

¹³C NMR (101 MHz, CDCl₃) δ 147.72, 144.78, 134.13, 133.20, 132.67, 131.20, 129.14, 127.13, 125.10, 122.41, 121.83, 110.25, 48.31, 47.85, 45.26, 28.03, 22.11, 10.73.

### B) Preparation of N-cyclopentyl-N-((1-ethyl-1,2,3,4-tetrahydroquinolin-6-yl)methyl)-3-nitrobenzenesulfon-amide (Compound 1)

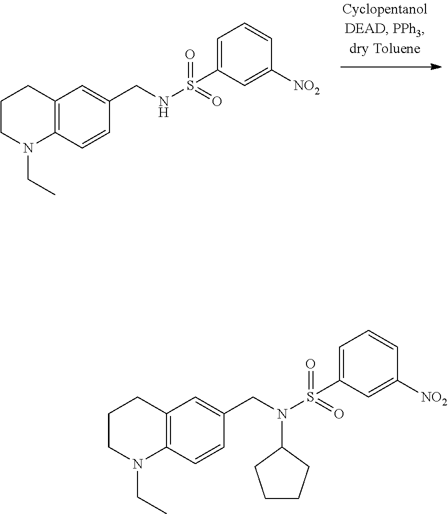 [SMILES: CCN1CCCc2cc(CNS(=O)(=O)c3cccc([N+](=O)[O-])c3)ccc21 ; CCN1CCCc2cc(CN(C3CCCC3)S(=O)(=O)c3cccc([N+](=O)[O-])c3)ccc21]

To a stirring solution of N-((1-ethyl-1,2,3,4-tetrahydroquinolin-6-yl)methyl)-3-nitrobenzenesulfonamide (722 mg, 2.102 mmol, 1 eq), PPh₃ (1652 mg, 6.306 mmol, 3 eq) and cyclopentanol (572 μL, 6.306 mmol, 3 eq) in anhydrous toluene, activated molecular sieve were added and the mixture was allowed to stir at 0° C. for 15 minutes. After a diethylazodicarboxylate solution (40% in toluene) was added dropwise under inert atmosphere (1528 μL, 8.390 mmol, 3 eq). The mixture was stirred at room temperature overnight. The mixture was evaporated under reduced pressure and then the desired product was purified by flash chromatography (Hexane/EtOAc 9:1) as a pale orange solid (930 mg, 100%).

LC-MS: t^{R} 17.98 min, (analytical HPLC, 10 to 90% acetonitrile (0.1% TFA) in water (0.1% TFA) over 20 min, flow rate of 1.0 mL/min). HRMS calculated: 444.19515 for C₂₃H₃₀N₃O₄S [M+H]⁺, found: 444.19452.

¹H NMR (400 MHz, CDCl₃) δ 8.38 (t, J=2.1 Hz, 1H), 8.32-8.25 (m, 1H), 8.02-7.95 (m, 1H), 7.59 (t, J=8.0 Hz, 1H), 6.86 (d, J=8.3 Hz, 1H), 6.74 (s, 1H), 6.39 (d, J=8.4 Hz, 1H), 4.37 (p, J=8.4 Hz, 1H), 4.25 (s, 2H), 3.27 (q, J=7.1 Hz, 2H), 3.21 (d, J=6.4 Hz, 2H), 2.60 (t, J=6.4 Hz, 2H), 1.88 (d, J=6.4 Hz, 2H), 1.84-1.70 (m, 3H), 1.68-1.57 (m, 2H), 1.57-1.46 (m, 4H), 1.09 (t, J=7.0 Hz, 3H).

¹³C NMR (101 MHz, CDCl₃) δ 148.30, 144.45, 132.73, 130.24, 129.35, 127.43, 126.49, 122.67, 110.44, 60.27, 48.66, 47.75, 45.72, 30.24, 28.46, 23.84, 22.52, 11.23.

According to the general procedure reported in FIG. 2 the following final compounds were prepared:

### 3-Bromo-N-cyclopentyl-N-((1-ethyl-1,2,3,4-tetrahydroquinolin-6-yl)methyl)-benzene-sulfonamide (Compound 3)

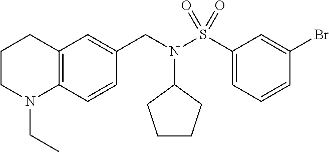 [SMILES: CCN1CCCc2cc(CN(C3CCCC3)S(=O)(=O)c3cccc(Br)c3)ccc21]

Purification (Hexane/EtOAc 92:8). Yield=82%. LC-MS: t^{R} 18.60 min, (analytical HPLC, 10 to 90% acetonitrile (0.1% TFA) in water (0.1% TFA) over 20 min, flow rate of 1.0 mL/min). HRMS calculated: 477.12059 for C₂₃H₃₀BrN₂O₂S [M+H]⁺, found: 477.12050. Calculated: 479.11854 for C₂₃H₃₀BrN₂O₂S [M+H]⁺, found: 479.11868.

¹H NMR (400 MHz, CDCl₃) δ 7.87-7.79 (m, 1H), 7.68 (dd, J=7.7, 2.2 Hz, 1H), 7.62 (dt, J=8.6, 2.1 Hz, 1H), 7.30 (td, J=8.0, 2.7 Hz, 1H), 6.98-6.89 (m, 1H), 6.84 (s, 1H), 6.48 (dd, J=8.5, 2.7 Hz, 1H), 4.31-4.18 (m, 3H), 3.31 (q, J=7.1 Hz, 2H), 3.23 (t, J=6.5 Hz, 2H), 2.68 (t, J=6.5 Hz, 2H), 1.92 (p, J=6.5 Hz, 2H), 1.74-1.61 (m, 2H), 1.61-1.50 (m, 2H), 1.50-1.37 (m, 4H), 1.12 (t, J=7.1 Hz, 3H).

¹³C NMR (101 MHz, CDCl₃) δ 144.42, 143.45, 135.03, 130.37, 130.18, 128.64, 126.59, 125.65, 124.21, 122.83, 122.50, 110.37, 59.75, 48.47, 47.46, 45.51, 29.61, 28.28, 23.57, 22.40, 10.96.

### N-cyclopentyl-N-((1-ethyl-1,2,3,4-tetrahydroquinolin-6-yl)methyl)-4-vinylbenzene-sulfonamide (Compound 4)

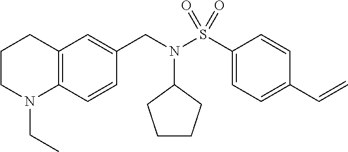 [SMILES: C=Cc1ccc(S(=O)(=O)N(Cc2ccc3c(c2)CCCN3CC)C2CCCC2)cc1]

Purification (Hexane/EtOAc 92:8). Yield=91%. LC-MS: t^{R} 18.34 min, (analytical HPLC, 10 to 90% acetonitrile (0.1% TFA) in water (0.1% TFA) over 20 min, flow rate of 1.0 mL/min). HRMS calculated: 425.22572 for C₂₅H₃₃N₂O₂S [M+H]⁺, found: 425.22558.

¹H NMR (400 MHz, CDCl₃) δ 7.73 (d, J=8.1 Hz, 2H), 7.46 (d, J=8.1 Hz, 2H), 7.06-6.94 (m, 1H), 6.88 (s, 1H), 6.73 (dd, J=17.6, 10.9 Hz, 1H), 6.50 (d, J=8.4 Hz, 1H), 5.86 (d, J=17.6 Hz, 1H), 5.40 (d, J=10.9 Hz, 1H), 4.24 (s, 2H), 4.19-3.99 (m, 1H), 3.31 (q, J=7.1 Hz, 2H), 3.22 (t, J=5.6 Hz, 2H), 2.68 (t, J=6.4 Hz, 2H), 1.93 (q, J=6.3 Hz, 2H), 1.79-1.58 (m, 4H), 1.58-1.47 (m, 2H), 1.47-1.35 (m, 2H), 1.11 (t, J=7.0 Hz, 3H).

¹³C NMR (101 MHz, CDCl₃) δ 144.28, 141.23, 140.30, 135.64, 128.47, 127.54, 126.58, 126.34, 124.98, 122.53, 116.95, 110.42, 59.61, 48.46, 47.37, 45.48, 29.41, 28.26, 23.54, 22.43, 10.87.

### N-cyclopentyl-N-((1-ethyl-1,2,3,4-tetrahydroquinolin-6-yl)methyl)-3-isopropyl-benzenesulfonamide (Compound 5)

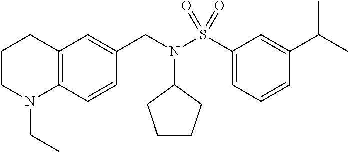 [SMILES: CCN1CCCc2cc(CN(C3CCCC3)S(=O)(=O)c3cccc(C(C)C)c3)ccc21]

Purification (Hexane/EtOAc 92:8). Yield=44%. LC-MS: t^{R} 19.16 min, (analytical HPLC, 10 to 90% acetonitrile (0.1% TFA) in water (0.1% TFA) over 20 min, flow rate of 1.0 mL/min). HRMS calculated: 441.25702 for C₂₆H₃₇N₂O₂S [M+H]⁺, found: 441.25732. Calculated: 463,23952 for C₂₆H₃₆N₂NaO₂S [M+Na]⁺, found: 463,23831.

¹H NMR (400 MHz, CDCl₃) δ 7.65 (s, 1H), 7.63 (dd, J=6.0, 2.7 Hz, 1H), 7.46-7.35 (m, 2H), 6.99 (dd, J=8.3, 2.2 Hz, 1H), 6.91 (d, J=2.2 Hz, 1H), 6.51 (d, J=8.4 Hz, 1H), 4.28-4.18 (m, 3H), 3.32 (q, J=7.1 Hz, 2H), 3.23 (t, J=5.7 Hz, 2H), 2.96 (hept, J=6.9 Hz, 1H), 2.70 (t, J=6.4 Hz, 2H), 1.93 (p, J=6.1 Hz, 2H), 1.67-1.57 (m, 2H), 1.55-1.47 (m, 2H), 1.46-1.34 (m, 4H), 1.26 (d, J=6.9 Hz, 6H), 1.12 (t, J=7.0 Hz, 3H).

¹³C NMR (101 MHz, CDCl₃) δ 149.97, 144.25, 141.16, 130.39, 128.88, 128.45, 126.32, 125.17, 125.12, 124.68, 122.48, 110.38, 59.61, 48.45, 47.27, 45.46, 34.17, 29.32, 28.28, 23.90, 23.56, 22.42, 10.89.

### N-cyclopentyl-N-((1-ethyl-1,2,3,4-tetrahydroquinolin-6-yl)methyl)naphthalene-2-sulfonamide (Compound 6)

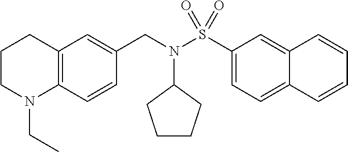 [SMILES: CCN1CCCc2cc(CN(C3CCCC3)S(=O)(=O)c3ccc4ccccc4c3)ccc21]

Purification (Hexane/EtOAc 93:7). Yield=61%. LC-MS: t^{R} 18.85 min, (analytical HPLC, 10 to 90% acetonitrile (0.1% TFA) in water (0.1% TFA) over 20 min, flow rate of 1.0 mL/min). HRMS calculated: 449.22572 for C₂₇H₃₃N₂O₂S [M+H]⁺, found: 449.22597.

¹H NMR (400 MHz, CDCl₃) δ 8.29 (s, 1H), 7.97-7.83 (m, 3H), 7.82-7.72 (m, 1H), 7.64-7.51 (m, 2H), 7.00 (d, J=8.7 Hz, 1H), 6.87 (d, J=2.6 Hz, 1H), 6.48 (dd, J=8.4, 2.2 Hz, 1H), 4.40-4.25 (m, 3H), 3.29 (q, J=2.2 Hz, 2H), 3.18 (t, J=5.6 Hz, 2H), 2.61 (t, J=6.4 Hz, 2H), 1.92-1.82 (m, 2H), 1.73-1.62 (m, 2H), 1.56-1.35 (m, 6H), 1.10 (t, J=2.1 Hz, 3H).

¹³C NMR (101 MHz, CDCl₃) δ 144.24, 138.32, 134.60, 132.27, 129.30, 129.06, 128.55, 128.45, 128.37, 127.87, 127.31, 126.50, 124.70, 122.74, 122.40, 110.29, 59.65, 48.39, 47.41, 45.44, 29.55, 28.18, 23.55, 22.34, 10.92.

### N-cyclopentyl-N-((1-ethyl-1,2,3,4-tetrahydroquinolin-6-yl)methyl)-4-(2-methoxy-ethyl)benzenesulfonamide (Compound 14)

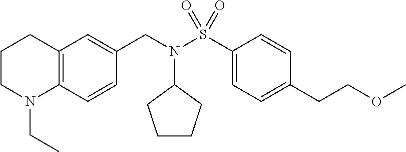 [SMILES: CCN1CCCc2cc(CN(C3CCCC3)S(=O)(=O)c3ccc(CCOC)cc3)ccc21]

Purification (Hexane/EtOAc 9:1). Yield=75%. LC-MS: t^{R} 18.20 min, (analytical HPLC, 10 to 90% acetonitrile (0.1% TFA) in water (0.1% TFA) over 20 min, flow rate of 1.0 mL/min). HRMS calculated: 479.23443 for C₂₆H₃₆N₂NaO₃S [M+H]⁺, found: 479.23481.

¹H NMR (400 MHz, CDCl₃) δ 7.64-7.58 (m, 2H), 7.39-7.30 (m, 2H), 6.98 (dd, J=8.4, 2.2 Hz, 1H), 6.91 (d, J=2.2 Hz, 1H), 6.51 (d, J=8.4 Hz, 1H), 4.29-4.19 (m, 3H), 3.32 (q, J=7.1 Hz, 2H), 3.23 (t, J=5.6 Hz, 2H), 2.70 (t, J=6.4 Hz, 2H), 2.65 (d, J=7.5 Hz, 2H), 2.07 (hept, J=7.7 Hz, 1H), 1.93 (p, J=6.1 Hz, 2H), 1.73-1.56 (m, 7H), 1.56-1.45 (m, 4H), 1.45-1.31 (m, 4H), 1.21-1.15 (m, 1H), 1.11 (t, J=7.0 Hz, 3H).

¹³C NMR (101 MHz, CDCl₃) δ 144.21, 143.56, 140.91, 132.70, 128.77, 128.44, 127.40, 126.27, 125.18, 124.51, 122.50, 110.38, 59.55, 48.46, 47.26, 47.22, 45.49, 41.93, 32.47, 29.28, 28.28, 25.03, 23.52, 22.41, 10.87.

### 4-cyano-N-cyclopentyl-N-((1-ethyl-1,2,3,4-tetrahydroquinolin-6-yl)methyl)benzene-sulfonamide (Compound 19)

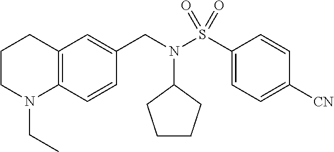 [SMILES: CCN1CCCc2cc(CN(C3CCCC3)S(=O)(=O)c3ccc(C#N)cc3)ccc21]

Purification (Hexane/EtOAc 9:1). Yield=66%. LC-MS: t^{R} 17.27 min, (analytical HPLC, 10 to 90% acetonitrile (0.1% TFA) in water (0.1% TFA) over 20 min, flow rate of 1.0 mL/min). HRMS calculated: 446,18782 for C₂₄H₃₂N₃NaO₂S [M+Na]⁺, found: 446, 18698.

¹H NMR (400 MHz, CDCl₃) δ 7.78 (d, J=8.3 Hz, 2H), 7.67 (d, J=8.4 Hz, 2H), 6.89 (dd, J=8.4, 2.4 Hz, 1H), 6.75 (d, J=2.3 Hz, 1H), 6.45 (d, J=8.4 Hz, 1H), 4.34-4.18 (m, 3H), 3.31 (q, J=7.0 Hz, 2H), 3.23 (t, J=5.6 Hz, 2H), 2.63 (t, J=6.3 Hz, 2H), 1.92 (p, J=6.1 Hz, 2H), 1.76-1.63 (m, 2H), 1.63-1.53 (m, 2H), 1.53-1.38 (m, 4H), 1.12 (t, J=7.0 Hz, 3H).

¹³C NMR (101 MHz, CDCl₃) δ 145.92, 144.40, 132.53, 128.76, 127.62, 126.80, 123.47, 122.35, 117.62, 115.44, 110.23, 59.85, 48.39, 47.47, 45.39, 29.71, 28.22, 23.43, 22.30, 10.82.

### N-cyclopentyl-N-((1-ethyl-1,2,3,4-tetrahydroquinolin-6-yl)methyl)-2-nitrobenzene-sulfonamide (Compound 20)

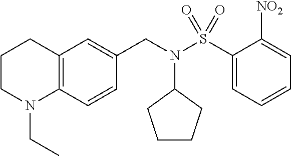 [SMILES: CCN1CCCc2cc(CN(C3CCCC3)S(=O)(=O)c3ccccc3[N+](=O)[O-])ccc21]

Purification (Hexane/EtOAc 85:15). Yield=72%. LC-MS: t^{R} 17.57 min, (analytical HPLC, 10 to 90% acetonitrile (0.1% TFA) in water (0.1% TFA) over 20 min, flow rate of 1.0 mL/min). HRMS calculated: 444.19515 for C₂₃H₃₀N₃O₄S [M+H]⁺, found: 444.19482. Calculated: 466,17765 for C₂₃H₂₉N₃NaO₄S [M+Na]⁺, found: 466,17715.

¹H NMR (400 MHz, CDCl₃) δ 7.69 (d, J=7.9 Hz, 1H), 7.58-7.50 (m, 2H), 7.41 (ddd, J=8.5, 6.1, 2.7 Hz, 1H), 6.89 (dd, J=8.5, 2.3 Hz, 1H), 6.77 (d, J=2.3 Hz, 1H), 6.38 (d, J=8.4 Hz, 1H), 4.46-4.20 (m, 3H), 3.28 (q, J=7.0 Hz, 2H), 3.19 (t, J=5.6 Hz, 2H), 2.57 (t, J=6.4 Hz, 2H), 1.98-1.76 (m, 4H), 1.62-1.46 (m, 6H), 1.08 (t, J=7.1 Hz, 3H).

¹³C NMR (101 MHz, CDCl₃) δ 147.65, 144.34, 135.11, 132.55, 131.27, 131.03, 128.98, 127.03, 123.79, 123.44, 122.33, 110.26, 59.94, 48.41, 47.63, 45.37, 29.66, 28.12, 23.56, 22.33, 10.79.

### Example 2

### A) Preparation of N-((1-ethyl-1,2,3,4-tetrahydroquinolin-6-yl)methyl)-4-nitrobenzenesulfonamide

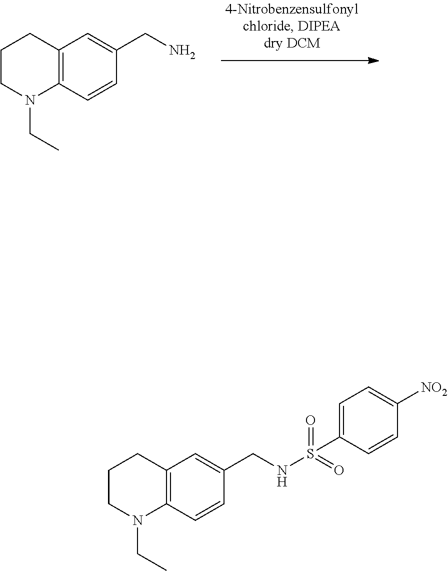 [SMILES: CCN1CCCc2cc(CN)ccc21 ; CCN1CCCc2cc(CNS(=O)(=O)c3ccc([N+](=O)[O-])cc3)ccc21]

A stirring solution of (1-ethyl-1,2,3,4-tetrahydroquinolin-6-yl)methanamine (570 mg, 3 mmol, 1 eq) and DIPEA (548 μL, 3.15 mmol, 1.05 eq) in anhydrous DCM (8 mL) was cooled down to 0° C. in an ice bath, then a solution of 4-nitrobenzenesulfonyl chloride (1183 mg, 3.15 mmol, 1.05 eq) in anhydrous DCM (2 mL) was added dropwise and the resulting mixture was allowed to stir overnight at room temperature. The organic solvent was evaporated under reduced pressure, the reaction was extracted in EtOAc and washed with HCl 0.1 M (2×30 mL) and NaHCO₃ sat. sol. (2×50 mL). Each aqueous aliquot was counter extracted with 2 aliquots of EtOAc. The organic phases were collected, dried over anhydrous sodium sulfate, filtered and volatiles evaporated under vacuum. The desired product was obtained after evaporation of the organic solvent as a pale-yellow powder (1070 mg, 95%) and was used in the next steps without any further purification.

LC-MS: t^{R} 10.12 min, (analytical HPLC, 10 to 90% acetonitrile (0.1% TFA) in water (0.1% TFA) over 20 min, flow rate of 1.0 mL/min). LRMS calculated: 376.13 for C₁₈H₂₂N₃O₄S [M+H]⁺, found: 376.50.

¹H NMR (400 MHz, CDCl₃) δ 8.39-8.16 (m, 2H), 8.03-7.86 (m, 2H), 6.77 (dd, J=8.4, 2.3 Hz, 1H), 6.63 (d, J=2.3 Hz, 1H), 6.40 (d, J=8.4 Hz, 1H), 4.82 (t, J=5.6 Hz, 1H), 4.06 (d, J=5.7 Hz, 2H), 3.28 (q, J=7.1 Hz, 2H), 3.21 (d, J=6.4 Hz, 2H), 2.58 (t, J=6.4 Hz, 2H), 1.87 (p, J=6.4 Hz, 2H), 1.09 (t, J=7.1 Hz, 3H).

¹³C NMR (101 MHz, CDCl₃) δ 146.52, 145.00, 129.31, 128.49, 127.32, 124.20, 122.61, 121.53, 110.34, 48.40, 47.47, 45.37, 28.17, 22.13, 10.77.

### B) Preparation of N-cyclopentyl-N-((1-ethyl-1,2,3,4-tetrahydroquinolin-6-yl)methyl)-4-nitrobenzenesulfonamide (Compound 2)

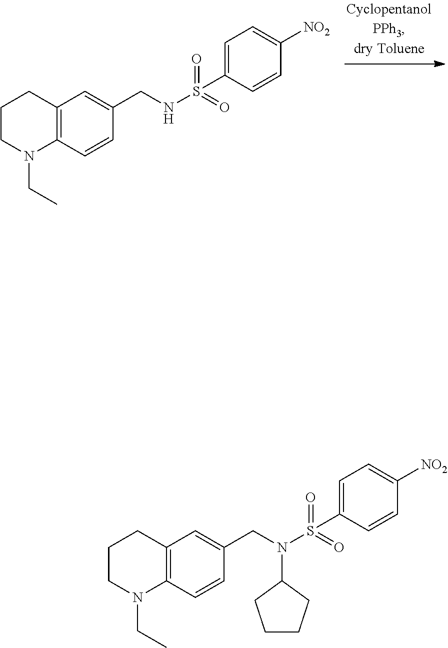 [SMILES: CCN1CCCc2cc(CNS(=O)(=O)c3ccc([N+](=O)[O-])cc3)ccc21 ; CCN1CCCc2cc(CN(C3CCCC3)S(=O)(=O)c3ccc([N+](=O)[O-])cc3)ccc21]

To a stirring solution of N-((1-ethyl-1,2,3,4-tetrahydroquinolin-6-yl)methyl)-4-nitrobenzenesulfonamide (1050 mg, 2.797 mmol, 1 eq), PPh₃ (2200 mg, 8.390 mmol, 3 eq) and cyclopentanol (762 μL, 8.390 mmol, 3 eq) in anhydrous toluene, activated molecular sieve were added and the mixture was allowed to stir at 0° C. for 15 minutes. After a diethylazodicarboxylate solution (40% in toluene) (1528 μL, 8.390 mmol, 3 eq) was added dropwise under inert atmosphere (1528 μL, 8.390 mmol, 3 eq). The mixture was stirred at room temperature overnight. The mixture was evaporated under reduced pressure and then the desired product was purified by flash chromatography (Hexane/EtOAc 7:3 to 1:1) as a pale-yellow liquid (1005 mg, 81%).

LC-MS: t^{R} 12.15 min, (analytical HPLC, 10 to 90% acetonitrile (0.1% TFA) in water (0.1% TFA) over 20 min, flow rate of 1.0 mL/min). HRMS calculated: 444.19515 for C₂₃H₃₀N₃O₄S [M+H]⁺, found: 444.19567.

¹H NMR (400 MHz, CDCl₃) δ 8.21 (d, J=8.4 Hz, 2H), 7.82 (d, J=8.3 Hz, 2H), 6.89 (d, J=8.3 Hz, 1H), 6.74 (s, 1H), 6.44 (d, J=8.4 Hz, 1H), 4.32 (p, J=8.5 Hz, 1H), 4.26 (s, 2H), 3.30 (q, J=7.1 Hz, 2H), 3.22 (t, J=5.7 Hz, 2H), 2.60 (t, J=6.4 Hz, 2H), 1.89 (p, J=6.0 Hz, 2H), 1.80-1.67 (m, 3H), 1.67-1.55 (m, 2H), 1.55-1.42 (m, 4H), 1.11 (t, J=7.1 Hz, 3H).

¹³C NMR (101 MHz, CDCl₃) δ 149.50, 147.59, 144.48, 128.92, 128.22, 127.01, 123.96, 123.31, 122.38, 110.25, 59.95, 48.41, 47.51, 45.41, 29.81, 28.24, 23.47, 22.29, 10.79.

### C) Preparation of 4-amino-N-cyclopentyl-N-((1-ethyl-1,2,3,4-tetrahydroquinolin-6-yl)methyl)benzenesulfonamide

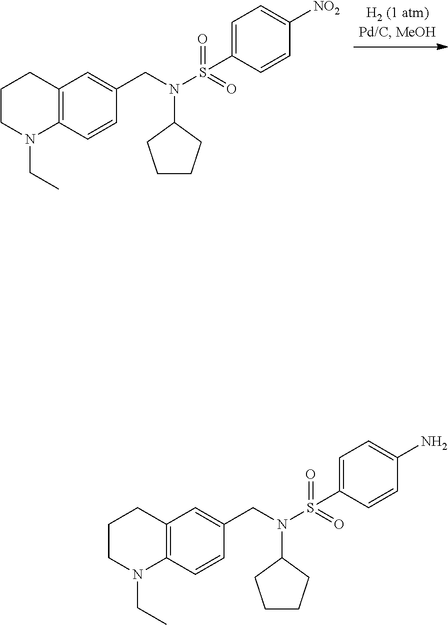 [SMILES: CCN1CCCc2cc(CN(C3CCCC3)S(=O)(=O)c3ccc([N+](=O)[O-])cc3)ccc21 ; CCN1CCCc2cc(CN(C3CCCC3)S(=O)(=O)c3ccc(N)cc3)ccc21]

A stirring solution of N-cyclopentyl-N-((1-ethyl-1,2,3,4-tetrahydroquinolin-6-yl)methyl)-4-nitrobenzenesulfonamide (990 mg, 2.223 mmol, 1 eq) in anhydrous MeOH (12 mL) was flushed with Nitrogen and then added with a suspension of Pd/C (10% w/w) (99, 10% in weight in respect to the substrate) in anhydrous MeOH (3 mL). The mixture was gently stirred under a Nitrogen atmosphere for 10 minutes. A flow of H₂ (g) was bubbled through the suspension at room temperature for 3 hours when conversion was complete. The mixture was filtered through a celite patch and eluted with a mixture of MeOH/THF (1:1). The solvents were evaporated under reduced pressure and to obtain a pale-yellow sticky oil (813 mg, 89%) which was used in the following synthetic steps without any other purification.

LC-MS: t^{R} 11.35 min, (analytical HPLC, 10 to 90% acetonitrile (0.1% TFA) in water (0.1% TFA) over 20 min, flow rate of 1.0 mL/min). Calculated: 414.59 for C₂₃H₃₂N₃O₂S [M+H]⁺, found: 414.49.

¹H NMR (400 MHz, CDCl₃) δ 8.02 (d, J=8.3 Hz, 2H), 7.57 (d, J=8.3 Hz, 2H), 6.87 (d, J=8.3 Hz, 1H), 6.69 (s, 1H), 6.43 (d, J=8.3 Hz, 1H), 5.93 (bs, 2H), 4.32 (p, J=8.5 Hz, 1H), 4.24 (s, 2H), 3.28 (q, J=7.1 Hz, 2H), 3.21 (t, J=5.7 Hz, 2H), 2.60 (t, J=6.4 Hz, 2H), 1.87 (p, J=6.0 Hz, 2H), 1.81-1.69 (m, 3H), 1.65-1.57 (m, 2H), 1.54-1.44 (m, 4H), 1.09 (t, J=7.1 Hz, 3H).

According to the above-mentioned protocol the following intermediate was prepared:

### 3-Amino-N-cyclopentyl-N-((1-ethyl-1,2,3,4-tetrahydroquinolin-6-yl)methyl)benzene-sulfonamide

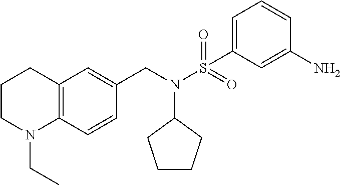 [SMILES: CCN1CCCc2cc(CN(C3CCCC3)S(=O)(=O)c3cccc(N)c3)ccc21]

Yield=80%. LC-MS: t^{R} 9.88 min, (analytical HPLC, 10 to 90% acetonitrile (0.1% TFA) in water (0.1% TFA) over 20 min, flow rate of 1.0 mL/min). Calculated: 414.22 for C₂₃H₃₂N₃O₂S [M+H]⁺, found: 414.27.

¹H NMR (400 MHz, CDCl₃) δ 8.37 (t, J=2.0 Hz, 1H), 8.31-8.26 (m, 1H), 7.99-7.94 (m, 1H), 7.59 (t, J=8.0 Hz, 1H), 6.84 (d, J=8.3 Hz, 1H), 6.75 (s, 1H), 6.34 (d, J=8.3 Hz, 1H), 6.74 (bs, 2H), 4.36 (p, J=8.4 Hz, 1H), 4.25 (s, 2H), 3.25 (q, J=7.0 Hz, 2H), 3.18 (d, J=6.5 Hz, 2H), 2.59 (t, J=6.4 Hz, 2H), 1.85 (d, J=6.5 Hz, 2H), 1.82-1.68 (m, 3H), 1.66-1.59 (m, 2H), 1.54-1.43 (m, 4H), 1.07 (t, J=7.0 Hz, 3H).

### D) Preparation of N-(4-(N-cyclopentyl-N-((1-ethyl-1,2,3,4-tetrahydroquinolin-6-yl)methyl)sulfamoyl)-phenyl)-2-phenylacetamide (Compound 8)

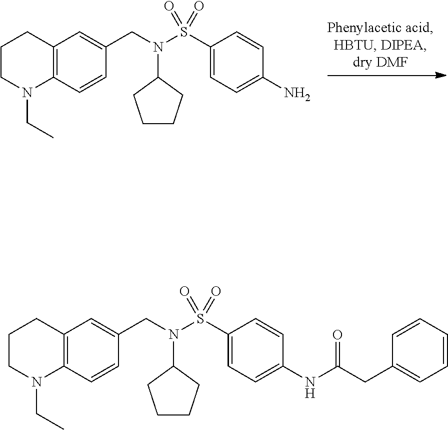 [SMILES: CCN1CCCc2cc(CN(C3CCCC3)S(=O)(=O)c3ccc(N)cc3)ccc21 ; CCN1CCCc2cc(CN(C3CCCC3)S(=O)(=O)c3ccc(NC(=O)Cc4ccccc4)cc3)ccc21]

Phenylacetic acid (25 mg, 0.182 mmol, 1.5 eq) and HBTU (69 mg, 0.182 mmol, 1.5 eq) were charged in a round bottom flask and then dissolved in anhydrous DMF (1 mL) in the presence of DIPEA (63 μL, 0.364 mmol, 3 eq). After 30 minutes of stirring at room temperature, a solution of 4-amino-N-cyclopentyl-N-((1-ethyl-1,2,3,4-tetrahydroquinolin-6-yl)methyl)benzenesulfonamide (50 mg, 0.121 mmol, 1 eq) in anhydrous DMF (0.5 mL) was added and the reaction was stirred at room temperature overnight. The mixture was extracted in EtOAc, washed with HCl 0.5M and NaHCO₃ sat. sol. twice and the aqueous solution counter extracted with one aliquot of EtOAc. The organic phases were collected, dried over anhydrous sodium sulfate, filtered and volatiles evaporated under vacuum to obtain a crude which was purified by column chromatography (Hex/EtOAc 7:3). The title compound was obtained as a pale-yellow foam (28 mg, 29%).

LC-MS: t^{R} 17.88 min, (analytical HPLC, 10 to 90% acetonitrile (0.1% TFA) in water (0.1% TFA) over 20 min, flow rate of 1.0 mL/min). HRMS calculated: 532,26284 for C₃₁H₃₈N₃O₃S [M+H]⁺, found: 532.26251.

¹H NMR (400 MHz, CDCl₃) δ 7.67 (d, J=8.5 Hz, 2H), 7.61 (s, 1H), 7.54 (d, J=8.6 Hz, 2H), 7.44-7.36 (m, 2H), 7.36-7.29 (m, 3H), 6.97 (dd, J=8.7, 2.0 Hz, 1H), 6.89 (s, 1H), 6.50 (d, J=8.4 Hz, 1H), 4.19 (d, J=8.2 Hz, 3H), 3.74 (s, 2H), 3.30 (q, J=7.1 Hz, 2H), 3.21 (t, J=5.6 Hz, 2H), 2.67 (t, J=6.5 Hz, 2H), 1.91 (p, J=6.1 Hz, 2H), 1.66-1.54 (m, 2H), 1.53-1.45 (m, 2H), 1.41-1.29 (m, 4H), 1.10 (t, J=7.0 Hz, 3H).

¹³C NMR (101 MHz, CDCl₃) δ 169.62, 141.35, 136.00, 134.10, 129.60, 129.37, 128.84, 128.34, 128.31, 127.90, 126.16, 122.63, 119.27, 110.59, 110.10, 59.52, 48.40, 47.21, 45.57, 44.87, 29.33, 28.20, 23.54, 22.27, 10.81.

According to the above-mentioned procedure the following compounds were prepared:

### 3-cyclopentyl-N-(4-(N-cyclopentyl-N-((1-ethyl-1,2,3,4-tetrahydroquinolin-6-yl)-methyl)sulfamoyl)phenyl)propanamide (Compound 9)

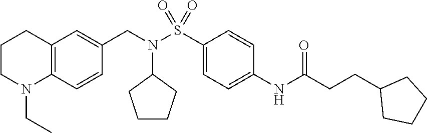 [SMILES: CCN1CCCc2cc(CN(C3CCCC3)S(=O)(=O)c3ccc(NC(=O)CCC4CCCC4)cc3)ccc21]

Purification (Hexane/EtOAc 8:2). Yield=40%. LC-MS: t^{R} 19.44 min, (analytical HPLC, 10 to 90% acetonitrile (0.1% TFA) in water (0.1% TFA) over 20 min, flow rate of 1.0 mL/min). HRMS calculated: 538.30979 for C₃₁H₄₄N₃O₃S [M+H]⁺, found: 538.30955.

¹H NMR (400 MHz, CDCl₃) δ 7.82 (s, 1H), 7.70 (d, J=8.6 Hz, 2H), 7.63 (d, J=8.6 Hz, 2H), 6.98 (dd, J=8.5, 2.4 Hz, 1H), 6.90 (d, J=2.3 Hz, 1H), 6.51 (d, J=8.5 Hz, 1H), 4.30-4.13 (m, 3H), 3.31 (q, J=7.1 Hz, 2H), 3.22 (t, J=5.6 Hz, 2H), 2.69 (t, J=6.4 Hz, 2H), 2.43-2.32 (m, 4H), 1.92 (p, J=6.2 Hz, 2H), 1.84-1.68 (m, 6H), 1.68-1.56 (m, 6H), 1.54-1.46 (m, 3H), 1.41-1.31 (m, 2H), 1.10 (t, J=7.0 Hz, 3H).

¹³C NMR (101 MHz, CDCl₃) δ 179.93, 172.35, 144.19, 141.84, 135.53, 128.37, 126.12, 125.15, 122.60, 119.24, 110.54, 59.53, 48.41, 47.25, 45.51, 39.77, 39.66, 37.16, 33.50, 32.58, 32.48, 31.73, 30.99, 28.25, 25.25, 25.21, 23.56, 22.34, 10.80.

### N-(3-(N-cyclopentyl-N-((1-ethyl-1,2,3,4-tetrahydroquinolin-6-yl)methyl)sulfamoyl)-phenyl)benzamide (Compound 11)

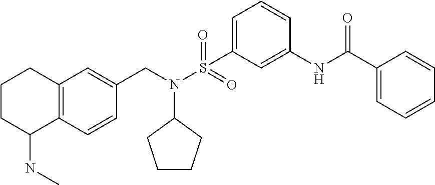 [SMILES: CNC1CCCc2cc(CN(C3CCCC3)S(=O)(=O)c3cccc(NC(=O)c4ccccc4)c3)ccc21]

Purification (Hexane/EtOAc 85:15). Yield=57%. LC-MS: t^{R} 17.02 min, (analytical HPLC, 10 to 90% acetonitrile (0.1% TFA) in water (0.1% TFA) over 20 min, flow rate of 1.0 mL/min). HRMS calculated: 518.24719 for C₃₀H₃₆N₃O₃S [M+H]⁺, found: 518.24698.

¹H NMR (400 MHz, CDCl₃) δ 8.70 (s, 1H), 8.36 (d, J=8.1 Hz, 1H), 8.04-7.95 (m, 2H), 7.80 (d, J=2.1 Hz, 1H), 7.60-7.40 (m, 5H), 6.81 (dd, J=8.5, 2.3 Hz, 1H), 6.74 (d, J=2.3 Hz, 1H), 6.32 (d, J=8.4 Hz, 1H), 4.28 (p, J=8.5 Hz, 1H), 4.14 (s, 2H), 3.20 (q, J=7.1 Hz, 2H), 3.13 (t, J=5.7 Hz, 2H), 2.50 (t, J=6.4 Hz, 2H), 1.87-1.74 (m, 2H), 1.69-1.54 (m, 2H), 1.54-1.39 (m, 2H), 1.39-1.20 (m, 4H), 1.04 (t, J=7.0 Hz, 3H).

¹³C NMR (101 MHz, CDCl₃) δ 166.13, 144.09, 140.98, 139.33, 134.30, 132.15, 129.78, 128.83, 128.25, 127.58, 126.23, 124.54, 123.95, 122.40, 122.13, 118.68, 110.32, 59.68, 48.28, 47.27, 45.37, 29.44, 28.05, 23.56, 22.20, 10.75.

### N-(3-(N-cyclopentyl-N-((1-ethyl-1,2,3,4-tetrahydroquinolin-6-yl)methyl)sulfamoyl)-phenyl)-2-phenylacetamide (Compound 12)

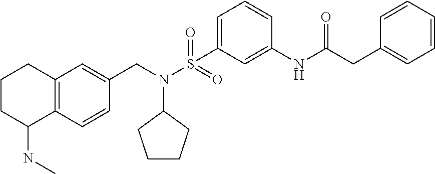 [SMILES: CNC1CCCc2cc(CN(C3CCCC3)S(=O)(=O)c3cccc(NC(=O)Cc4ccccc4)c3)ccc21]

Purification (Hexane/EtOAc 85:15). Yield=35%. LC-MS: t^{R} 17.18 min, (analytical HPLC, 10 to 90% acetonitrile (0.1% TFA) in water (0.1% TFA) over 20 min, flow rate of 1.0 mL/min). HRMS calculated: 532.26284 for C₃₁H₃₈N₃O₃S [M+H]⁺, found: 532.26303.

¹H NMR (400 MHz, CDCl₃) δ 8.07 (s, 1H), 7.99 (d, J=8.1 Hz, 1H), 7.70 (s, 1H), 7.47 (d, J=7.9 Hz, 1H), 7.40-7.27 (m, 6H), 6.96 (dd, J=8.5, 2.2 Hz, 1H), 6.87 (d, J=2.2 Hz, 1H), 6.47 (d, J=8.4 Hz, 1H), 4.30-4.15 (m, 3H), 3.72 (s, 2H), 3.29 (q, J=7.1 Hz, 2H), 3.21 (t, J=5.6 Hz, 2H), 2.62 (t, J=6.4 Hz, 2H), 1.89 (p, J=6.0 Hz, 2H), 1.68-1.54 (m, 2H), 1.54-1.42 (m, 2H), 1.42-1.30 (m, 4H), 1.10 (t, J=7.0 Hz, 3H).

¹³C NMR (101 MHz, CDCl₃) δ 169.80, 143.95, 141.19, 138.89, 134.41, 129.81, 129.59, 129.13, 128.31, 127.60, 126.20, 125.25, 123.49, 122.73, 122.22, 118.07, 110.76, 59.66, 48.35, 47.41, 45.67, 44.62, 29.37, 28.09, 23.57, 22.14, 10.80.

### 3-cyclopentyl-N-(3-(N-cyclopentyl-N-((1-ethyl-1,2,3,4-tetrahydroquinolin-6-yl)-methyl)-sulfamoyl)phenyl)34ropenamide (Compound 13)

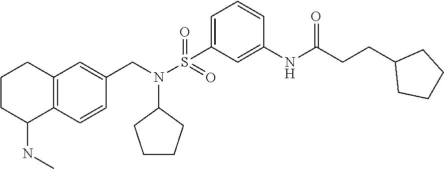 [SMILES: CNC1CCCc2cc(CN(C3CCCC3)S(=O)(=O)c3cccc(NC(=O)CCC4CCCC4)c3)ccc21]

Purification (Hexane/EtOAc 8:2). Yield=26%. LC-MS: t^{R} 17.25 min, (analytical HPLC, 10 to 90% acetonitrile (0.1% TFA) in water (0.1% TFA) over 20 min, flow rate of 1.0 mL/min). HRMS calculated: 538.30979 for C₃₁H₄₄N₃O₃S [M+H]⁺, found: 538.30997.

¹H NMR (400 MHz, CDCl₃) δ 8.08 (d, J=8.1 Hz, 1H), 7.89 (s, 1H), 7.64 (s, 1H), 7.47 (d, J=7.9 Hz, 1H), 7.39 (t, J=8.0 Hz, 1H), 6.95 (dd, J=8.4, 2.2 Hz, 1H), 6.84 (d, J=2.1 Hz, 1H), 6.47 (d, J=8.4 Hz, 1H), 4.32-4.13 (m, 3H), 3.30 (q, J=7.0 Hz, 2H), 3.21 (t, J=5.6 Hz, 2H), 2.64 (t, J=6.4 Hz, 2H), 2.39 (t, J=6.5 Hz, 2H), 1.90 (p, J=6.1 Hz, 2H), 1.82-1.70 (m, 6H), 1.67-1.56 (m, 6H), 1.54-1.46 (m, 4H), 1.42-1.33 (m, 3H), 1.10 (t, J=7.0 Hz, 3H).

¹³C NMR (101 MHz, CDCl₃) δ 172.25, 144.24, 141.32, 139.09, 129.81, 128.44, 126.36, 124.80, 123.40, 122.47, 121.97, 117.96, 110.37, 59.68, 48.43, 47.41, 45.47, 39.85, 37.06, 32.62, 31.78, 29.45, 28.23, 25.28, 23.57, 22.36, 10.81.

### E) Preparation of N-(4-(N-cyclopentyl-N-((1-ethyl-1,2,3,4-tetrahydroquinolin-6-yl)methyl)sulfamoyl)phenyl)acrylamide (Compound 25)

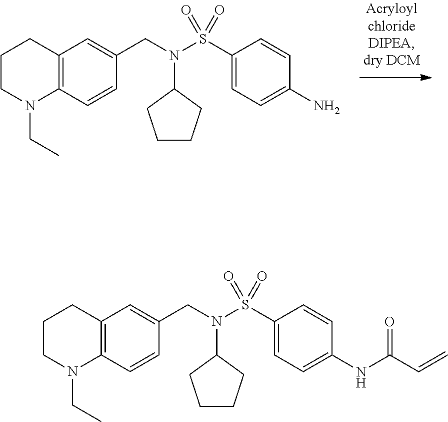 [SMILES: CCN1CCCc2cc(CN(C3CCCC3)S(=O)(=O)c3ccc(N)cc3)ccc21 ; C=CC(=O)Nc1ccc(S(=O)(=O)N(Cc2ccc3c(c2)CCCN3CC)C2CCCC2)cc1]

To a stirring solution of 4-amino-N-cyclopentyl-N-((1-ethyl-1,2,3,4-tetrahydroquinolin-6-yl)methyl)benzenesulfona-mide (50 mg, 0.121 mmol, 1 eq) and DIPEA (63 μL, 0.364 mmol, 3 eq) in anhydrous DCM (2 mL), a solution of acryloyl chloride (20 μL, 0.242 mmol, 2 eq) in anhydrous DCM (0.5 mL) was added dropwise at 0° C. The resulting solution was allowed to stir at room temperature for 4 hours when conversion was complete. The mixture was extracted in EtOAc and washed with HCl 0.5M. The aqueous phase was counter extracted with one aliquot of EtOAc.

The organic phases were collected, dried over anhydrous sodium sulfate, filtered and volatiles evaporated under vacuum to obtain a crude mixture which was purified by column chromatography (Hexane/EtOAc 7:3). The product was obtained as an off-white solid (49 mg, 86%).

LC-MS: t^{R} 16.30 min, (analytical HPLC, 10 to 90% acetonitrile (0.1% TFA) in water (0.1% TFA) over 20 min, flow rate of 1.0 mL/min). HRMS calculated: 468.23154 for C₂₆H₃₄N₃O₃S [M+H]⁺, found: 468.23112.

¹H NMR (400 MHz, CDCl₃) δ 8.53 (s, 1H), 7.74 (d, J=8.5 Hz, 2H), 7.69 (d, J=8.5 Hz, 2H), 6.98 (dd, J=8.4, 2.2 Hz, 1H), 6.89 (d, J=2.2 Hz, 1H), 6.51 (d, J=8.4 Hz, 1H), 6.44 (dd, J=16.9, 1.7 Hz, 1H), 6.33 (dd, J=16.8, 9.9 Hz, 1H), 5.73 (dd, J=9.9, 1.7 Hz, 1H), 4.27-4.14 (m, 3H), 3.29 (q, J=7.1 Hz, 2H), 3.21 (t, J=5.6 Hz, 2H), 2.67 (t, J=6.4 Hz, 2H), 1.90 (p, J=6.1 Hz, 2H), 1.66-1.55 (m, 2H), 1.55-1.44 (m, 2H), 1.42-1.31 (m, 4H), 1.10 (t, J=7.0 Hz, 3H).

¹³C NMR (101 MHz, CDCl₃) δ 164.26, 144.07, 142.05, 135.62, 130.98, 128.68, 128.26, 128.24, 126.11, 125.32, 122.73, 119.70, 110.76, 59.60, 48.37, 47.35, 45.65, 29.36, 28.18, 23.57, 22.21, 10.84.

According to the above-mentioned procedure the following compounds were prepared:

### N-(3-(N-cyclopentyl-N-((1-ethyl-1,2,3,4-tetrahydroquinolin-6-yl)methyl)sulfamoyl)-phenyl)acrylamide (Compound 17)

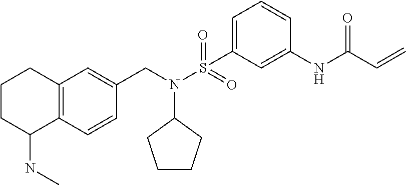 [SMILES: C=CC(=O)Nc1cccc(S(=O)(=O)N(Cc2ccc3c(c2)CCCC3NC)C2CCCC2)c1]

Purification (Hexane/EtOAc 9:1). Yield: 32%. LC-MS: t^{R} 16.38 min, (analytical HPLC, 10 to 90% acetonitrile (0.1% TFA) in water (0.1% TFA) over 20 min, flow rate of 1.0 mL/min). HRMS calculated: 468.23154 for C₂₆H₃₄N₃O₃S [M+H]⁺, found: 468.23123. HRMS calculated: 490,21403 for C₂₆H₃₃N₃NaO₃S [M+Na]⁺, found: 490,21350.

¹H NMR (400 MHz, CDCl₃) δ 8.23 (d, J=8.0 Hz, 1H), 8.16 (s, 1H), 7.63 (s, 1H), 7.49 (d, J=8.0 Hz, 1H), 7.42 (t, J=7.8 Hz, 1H), 6.97-6.86 (m, 1H), 6.81 (s, 1H), 6.53-6.37 (m, 2H), 6.30 (dd, J=16.8, 10.2 Hz, 1H), 5.77 (d, J=10.0 Hz, 1H), 4.33-4.13 (m, 3H), 3.28 (q, J=7.0 Hz, 2H), 3.20 (t, J=5.8 Hz, 2H), 2.61 (t, J=6.4 Hz, 2H), 1.88 (p, J=6.1 Hz, 2H), 1.76-1.58 (m, 2H), 1.58-1.46 (m, 2H), 1.46-1.32 (m, 4H), 1.09 (t, J=7.0 Hz, 3H).

¹³C NMR (101 MHz, CDCl₃) δ 163.99, 144.33, 141.37, 139.05, 131.17, 129.90, 128.45, 128.30, 126.43, 124.59, 123.58, 122.53, 122.13, 118.22, 110.40, 59.79, 48.43, 47.53, 45.49, 29.55, 28.23, 23.62, 22.37, 10.92.

### 2-bromo-N-(3-(N-cyclopentyl-N-((1-ethyl-1,2,3,4-tetrahydroquinoin-6-yl)methyl)-sulfamoyl)phenyl)acetamide (Compound 18)

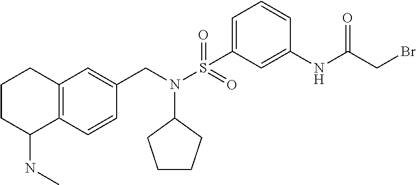 [SMILES: CNC1CCCc2cc(CN(C3CCCC3)S(=O)(=O)c3cccc(NC(=O)CBr)c3)ccc21]

Purification (Hexane/EtOAc 8:2). Yield=20%. LC-MS: t^{R} 16.68 min, (analytical HPLC, 10 to 90% acetonitrile (0.1% TFA) in water (0.1% TFA) over 20 min, flow rate of 1.0 mL/min). HRMS calculated: 534.14205 for C₂₅H₃₃BrN₃O₃S [M+H]⁺, found: 534.14262. HRMS calculated: 536.14000 for C₂₅H₃₃BrN₃O₃S [M+H]⁺, found: 536.14032.

¹H NMR (400 MHz, CDCl₃) δ 8.60 (s, 1H), 7.88 (d, J=8.1 Hz, 1H), 7.82 (s, 1H), 7.54 (d, J=8.0 Hz, 1H), 7.42 (t, J=8.0 Hz, 1H), 6.97 (dd, J=8.4, 2.3 Hz, 1H), 6.88 (s, 1H), 6.56 (d, J=8.3 Hz, 1H), 4.24 (s, 2H), 4.19 (s, 2H), 4.18-4.11 (m, 2H), 3.32 (q, J=7.1 Hz, 2H), 3.24 (t, J=5.7 Hz, 2H), 2.67 (t, J=6.5 Hz, 2H), 1.91 (p, J=6.1 Hz, 4H), 1.72-1.61 (m, 2H), 1.56-1.47 (m, 2H), 1.46-1.36 (m, 4H), 1.12 (t, J=7.1 Hz, 3H).

¹³C NMR (101 MHz, CDCl₃) δ 164.30, 152.30, 143.48, 141.94, 137.52, 129.70, 128.40, 126.28, 125.76, 123.37, 123.13, 118.37, 111.28, 59.59, 48.21, 47.27, 45.93, 42.89, 29.37, 29.05, 23.41, 22.99, 10.64.

### N-(3-(N-cyclopentyl-N-((1-ethyl-1,2,3,4-tetrahydroquinolin-6-yl)methyl)sulfamoyl)-phenyl)propionamide (Compound 21)

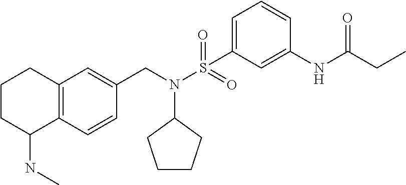 [SMILES: CCC(=O)Nc1cccc(S(=O)(=O)N(Cc2ccc3c(c2)CCCC3NC)C2CCCC2)c1]

Purification (Hexane/DCM/EtOAc 5:4:1). Yield=53%. LC-MS: t^{R} 16.42 min, (analytical HPLC, 10 to 90% acetonitrile (0.1% TFA) in water (0.1% TFA) over 20 min, flow rate of 1.0 mL/min). HRMS calculated: 470.24719 for C₂₆H₃₆N₃O₃S [M+H]⁺, found: 470.24689.

¹H NMR (400 MHz, CDCl₃) δ 8.14 (d, J=8.1 Hz, 1H), 8.08 (s, 1H), 7.67 (t, J=2.0 Hz, 1H), 7.47 (d, J=8.2 Hz, 1H), 7.39 (t, J=8.0 Hz, 1H), 6.95 (dd, J=8.4, 2.3 Hz, 1H), 6.84 (d, J=2.2 Hz, 1H), 6.46 (d, J=8.4 Hz, 1H), 4.31-4.15 (m, 3H), 3.30 (q, J=7.0 Hz, 2H), 3.21 (t, J=5.6 Hz, 2H), 2.63 (t, J=6.3 Hz, 2H), 2.40 (q, J=7.6 Hz, 2H), 1.95-1.85 (m, 2H), 1.69-1.56 (m, 2H), 1.56-1.45 (m, 2H), 1.44-1.31 (m, 4H), 1.23 (t, J=7.5 Hz, 3H), 1.10 (t, J=7.0 Hz, 3H).

¹³C NMR (101 MHz, CDCl₃) δ 172.83, 144.31, 141.23, 139.31, 129.82, 128.38, 126.32, 124.80, 123.42, 122.50, 121.77, 117.93, 110.38, 59.72, 48.43, 47.53, 45.47, 30.66, 29.44, 28.24, 23.59, 22.37, 10.88, 9.64.

### N-cyclopentyl-N-((1-ethyl-1,2,3,4-tetrahydroquinolin-6-yl)methyl)-3-(methylsulfon-amido)benzenesulfonamide (Compound 22)

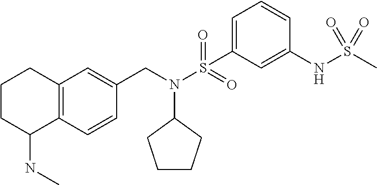 [SMILES: CNC1CCCc2cc(CN(C3CCCC3)S(=O)(=O)c3cccc(NS(C)(=O)=O)c3)ccc21]

Purification (Hexane/EtOAc 75:25). Yield=58%. LC-MS: t^{R} 17.06 min, (analytical HPLC, 10 to 90% acetonitrile (0.1% TFA) in water (0.1% TFA) over 20 min, flow rate of 1.0 mL/min). HRMS calculated: 492,19852 for C₂₄H₃₄N₃O₄S₂[M+H]⁺, found: 492,22903.

¹H NMR (400 MHz, CDCl₃) δ 7.87 (dt, J=7.5, 1.7 Hz, 1H), 7.79 (t, J=1.9 Hz, 1H), 7.59-7.48 (m, 2H), 6.98 (dd, J=8.4, 2.3 Hz, 1H), 6.90 (d, J=2.3 Hz, 1H), 6.51 (d, J=8.4 Hz, 1H), 4.34-4.15 (m, 3H), 3.38 (s, 3H), 3.32 (q, J=7.0 Hz, 2H), 3.23 (t, J=5.7 Hz, 2H), 2.70 (t, J=6.4 Hz, 2H), 1.98-1.88 (m, 2H), 1.69-1.59 (m, 2H), 1.56-1.47 (m, 2H), 1.40 (q, J=4.9 Hz, 4H), 1.12 (t, J=7.0 Hz, 3H).

¹³C NMR (101 MHz, CDCl₃) δ 144.39, 142.98, 134.20, 134.19, 130.23, 129.59, 128.92, 128.53, 126.43, 124.49, 122.57, 110.47, 59.82, 48.44, 47.36, 45.46, 42.89, 29.38, 28.23, 23.48, 22.39, 10.89.

### N-cyclopentyl-N-((1-ethyl-1,2,3,4-tetrahydroquinolin-6-yl)methyl)-4-(N-(methyl-sulfonyl)methylsulfonamido)benzenesulfonamide (Compound 24)

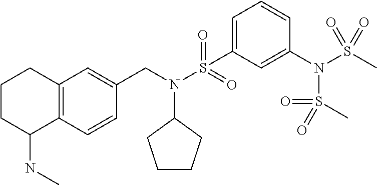 [SMILES: CNC1CCCc2cc(CN(C3CCCC3)S(=O)(=O)c3cccc(N(S(C)(=O)=O)S(C)(=O)=O)c3)ccc21]

Purification (Hexane/EtOAc 75:25). Yield=40%. LC-MS: t^{R} 17.06 min, (analytical HPLC, 10 to 90% acetonitrile (0.1% TFA) in water (0.1% TFA) over 20 min, flow rate of 1.0 mL/min). HRMS calculated: 570.17607 for C₂₅H₃₆N₃O₆S₃[M+H]⁺, found: 570.17644.

¹H NMR (400 MHz, CDCl₃) δ 7.85 (d, J=8.6 Hz, 2H), 7.44 (d, J=8.6 Hz, 2H), 6.94 (dd, J=8.4, 2.3 Hz, 1H), 6.90 (d, J=2.3 Hz, 1H), 6.51 (d, J=8.3 Hz, 1H), 4.32-4.16 (m, 3H), 3.41 (s, 6H), 3.32 (q, J=7.1 Hz, 2H), 3.23 (t, J=5.7 Hz, 2H), 2.71 (t, J=6.4 Hz, 2H), 2.00-1.86 (m, 2H), 1.72-1.61 (m, 2H), 1.57-1.50 (m, 2H), 1.50-1.37 (m, 4H), 1.12 (t, J=7.0 Hz, 3H).

¹³C NMR (101 MHz, CDCl₃) δ 144.44, 143.61, 136.64, 131.33, 128.55, 128.49, 126.42, 124.40, 122.64, 110.52, 59.87, 48.46, 47.64, 45.49, 42.89, 29.58, 28.27, 23.53, 22.43, 10.87.

### 2-bromo-N-(4-(N-cyclopentyl-N-((1-ethyl-1,2,3,4-tetrahydroquinoin-6-yl)methyl)-sulfamoyl)phenyl)acetamide (Compound 26)

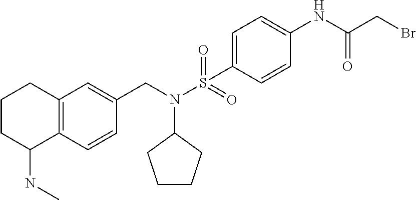 [SMILES: CNC1CCCc2cc(CN(C3CCCC3)S(=O)(=O)c3ccc(NC(=O)CBr)cc3)ccc21]

Purification (Hexane/DCM/EtOAc 5:4:1). Yield=22%. LC-MS: t^{R} 16.79 min, (analytical HPLC, 10 to 90% acetonitrile (0.1% TFA) in water (0.1% TFA) over 20 min, flow rate of 1.0 mL/min). HRMS calculated: 534.14205 for C₂₅H₃₃BrN₃O₃S [M+H]⁺, found: 534.14186. HRMS calculated: 536.14000 for C₂₅H₃₃BrN₃O₃S [M+H]⁺, found: 536.13979.

¹H NMR (400 MHz, CDCl₃) δ 8.46 (s, 1H), 7.79-7.72 (m, 2H), 7.71-7.62 (m, 2H), 6.97 (dd, J=8.4, 2.3 Hz, 1H), 6.89 (d, J=2.3 Hz, 1H), 6.51 (d, J=8.4 Hz, 1H), 4.30-4.12 (m, 5H), 3.32 (q, J=7.0 Hz, 2H), 3.23 (t, J=5.7 Hz, 2H), 2.69 (t, J=6.4 Hz, 2H), 1.93 (p, J=6.1 Hz, 2H), 1.65-1.57 (m, 2H), 1.57-1.45 (m, 2H), 1.45-1.34 (m, 4H), 1.12 (t, J=7.0 Hz, 3H).

¹³C NMR (101 MHz, CDCl₃) δ 164.26, 144.30, 140.24, 137.34, 128.54, 128.44, 126.31, 124.97, 122.62, 119.65, 110.54, 59.64, 48.48, 47.39, 45.55, 43.01, 29.44, 28.29, 23.57, 22.41, 10.90.

### N-(4-(N-cyclopentyl-N-((1-ethyl-1,2,3,4-tetrahydroquinolin-6-yl)methyl)sulfamoyl)-phenyl)propionamide (Compound 27)

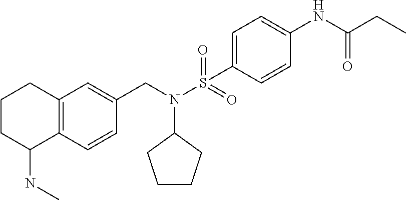 [SMILES: CCC(=O)Nc1ccc(S(=O)(=O)N(Cc2ccc3c(c2)CCCC3NC)C2CCCC2)cc1]

Purification (Hexane/EtOAc 7:3). Yield=52%. LC-MS: t^{R} 16.69 min, (analytical HPLC, 10 to 90% acetonitrile (0.1% TFA) in water (0.1% TFA) over 20 min, flow rate of 1.0 mL/min). HRMS calculated: 470.24719 for C₂₆H₃₆N₃O₃S [M+H]⁺, found: 470.24683.

¹H NMR (400 MHz, CDCl₃) δ 8.16 (s, 1H), 7.80-7.63 (m, 4H), 6.98 (dd, J=8.5, 2.3 Hz, 1H), 6.89 (d, J=2.2 Hz, 1H), 6.50 (d, J=8.4 Hz, 1H), 4.21 (s, 3H), 3.30 (q, J=7.1 Hz, 2H), 3.21 (t, J=5.6 Hz, 2H), 2.67 (t, J=6.4 Hz, 2H), 2.38 (q, J=7.5 Hz, 2H), 1.90 (p, J=6.1 Hz, 2H), 1.66-1.55 (m, 2H), 1.53-1.45 (m, 2H), 1.43-1.32 (m, 4H), 1.19 (t, J=7.5 Hz, 3H), 1.10 (t, J=7.0 Hz, 3H).

¹³C NMR (101 MHz, CDCl₃) δ 173.03, 144.28, 142.20, 135.22, 128.25, 128.19, 126.08, 125.00, 122.52, 119.29, 110.42, 59.56, 48.38, 47.34, 45.44, 30.69, 29.31, 28.24, 23.54, 22.34, 10.83, 9.56.

### N-cyclopentyl-N-((1-ethyl-1,2,3,4-tetrahydroquinolin-6-yl)methyl)-4-(trifluoromethyl-sulfonamido)benzenesulfonamide (Compound 30)

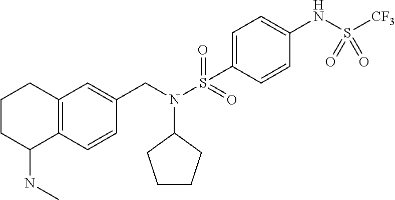 [SMILES: CNC1CCCc2cc(CN(C3CCCC3)S(=O)(=O)c3ccc(NS(=O)(=O)C(F)(F)F)cc3)ccc21]

Purification (Hexane/EtOAc 6:4). Yield=32%. LC-MS: t^{R} 16.33 min, (analytical HPLC, 10 to 90% acetonitrile (0.1% TFA) in water (0.1% TFA) over 20 min, flow rate of 1.0 mL/min). HRMS calculated: 546.17026 for C₂₄H₃₁F₃N₃O₄S₂ [M+H]⁺, found: 546.16990.

¹H NMR (400 MHz, CDCl₃) δ 7.70 (d, J=8.3 Hz, 2H), 7.30-7.22 (m, 2H), 6.94 (d, J=8.5 Hz, 1H), 6.87 (s, 1H), 6.52 (d, J=8.4 Hz, 1H), 5.86-5.52 (m, 1H), 4.29-4.14 (m, 3H), 3.32 (q, J=7.1 Hz, 2H), 3.23 (t, J=5.6 Hz, 2H), 2.67 (t, J=6.5 Hz, 2H), 1.92 (p, J=6.1 Hz, 2H), 1.67-1.56 (m, 2H), 1.54-1.46 (m, 2H), 1.46-1.34 (m, 5H), 1.11 (t, J=7.0 Hz, 3H).

¹³C NMR (101 MHz, CDCl₃) δ 143.97, 139.56, 138.28, 128.64, 128.39, 126.23, 125.52, 123.14, 122.13, 111.18, 59.71, 48.44, 47.37, 45.71, 31.73, 29.45, 28.13, 23.59, 22.79, 22.19, 14.26, 10.65.

### Example 3

### A) Preparation of N-cyclopentyl-N-((1-ethyl-1,2,3,4-tetrahydroquinolin-6-yl)methyl)-4-(furan-2-ylmethyl)benzenesulfonamide (Compound 10)

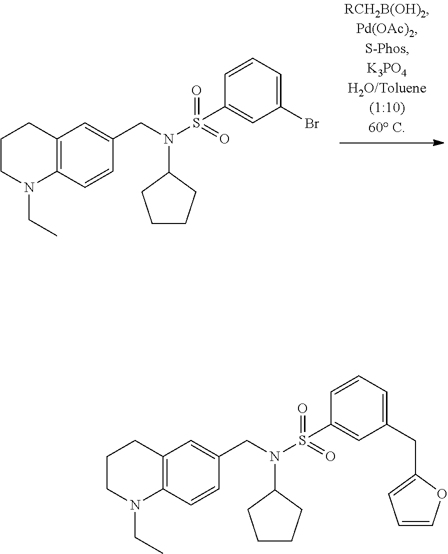 [SMILES: CCN1CCCc2cc(CN(C3CCCC3)S(=O)(=O)c3cccc(Br)c3)ccc21 ; CCN1CCCc2cc(CN(C3CCCC3)S(=O)(=O)c3cccc(Cc4ccco4)c3)ccc21]

3-Bromo-N-cyclopentyl-N-((1-ethyl-1,2,3,4-tetrahydroquinolin-6-yl)methyl)-benzenesulfonamide (96 mg, 0.2 mmol, 1 eq), K₃PO₄ (84 mg, 0.4 mmol, 2 eq) and (furan-2-ylmethyl)boronic acid (124 mg, 0.6 mmol, 3 eq) were charged in a vial and a mixture of water and toluene (3. mL, 1:10) was added. The mixture was allowed to stir at room temperature for 20 minutes while different cycles of vacuum and Argon flush were performed. Pd(OAc)₂ (6 mg, 0.026 mmol, 13% mol) and 2-Dicyclohexylphosphino-2′,6′-dimethoxybiphenyl (S-Phos) (22 mg, 0.052 mmol, 26% mol) were added under Argon atmosphere and the resulting mixture was stirred at 60° C. for 6 hours. The mixture was diluted with EtOAc and washed with water twice. The aqueous layers were combined and counter extracted with one aliquot of EtOAc. The organic phases were collected, dried over anhydrous sodium sulfate, filtered and volatiles evaporated under vacuum to obtain a crude which was purified by column chromatography (Hex/EtOAc 94:6). Then, the title compound was obtained as a pale-yellow solid (66 mg, 69%).

LC-MS: t^{R} 16.33 min, (analytical HPLC, 10 to 90% acetonitrile (0.1% TFA) in water (0.1% TFA) over 20 min, flow rate of 1.0 mL/min). HRMS calculated: 479.23629 for C₂₈H₃₅N₂O₃S [M+H]⁺, found: 479.23667.

¹H NMR (400 MHz, CDCl₃) δ 7.65 (d, J=5.6 Hz, 2H), 7.39 (d, J=4.6 Hz, 2H), 7.32 (d, J=1.8 Hz, 1H), 6.97 (dd, J=8.5, 2.3 Hz, 1H), 6.89 (d, J=2.2 Hz, 1H), 6.52 (d, J=8.4 Hz, 1H), 6.30 (t, J=2.5 Hz, 1H), 6.03 (d, J=3.1 Hz, 1H), 4.30-4.16 (m, 3H), 4.00 (s, 2H), 3.32 (q, J=7.1 Hz, 2H), 3.23 (t, J=5.6 Hz, 2H), 2.69 (t, J=6.4 Hz, 2H), 1.93 (p, J=6.1 Hz, 2H), 1.61-1.53 (m, 2H), 1.53-1.45 (m, 2H), 1.42-1.30 (m, 4H), 1.12 (t, J=7.0 Hz, 3H).

¹³C NMR (101 MHz, CDCl₃) δ 153.37, 144.03, 141.93, 141.34, 139.45, 132.49, 129.12, 128.45, 127.40, 126.29, 125.40, 122.69, 110.68, 110.51, 110.12, 106.90, 59.59, 48.43, 47.24, 45.66, 34.32, 29.29, 28.19, 23.53, 22.27, 10.85.

### N-cyclopentyl-N-((1-ethyl-1,2,3,4-tetrahydroquinolin-6-yl)methyl)-3-isobutyl-benzenesulfonamide (Compound 7)

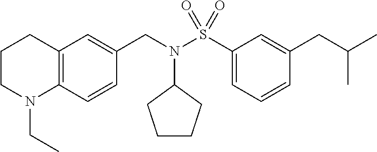 [SMILES: CCN1CCCc2cc(CN(C3CCCC3)S(=O)(=O)c3cccc(CC(C)C)c3)ccc21]

Purification (Hexane/EtOAc 95:5). Yield=82%. LC-MS: t^{R} 20.07 min, (analytical HPLC, 10 to 90% acetonitrile (0.1% TFA) in water (0.1% TFA) over 20 min, flow rate of 1.0 mL/min). HRMS calculated: 455.27267 for C₂₇H₃₉N₂O₂S [M+H]⁺, found: 455.27228.

¹H NMR (400 MHz, CDCl₃) δ 7.62 (d, J=7.6 Hz, 1H), 7.58 (s, 1H), 7.36 (t, J=7.7 Hz, 1H), 7.30 (d, J=7.5 Hz, 1H), 6.98 (d, J=8.3 Hz, 1H), 6.91 (s, 1H), 6.51 (d, J=8.4 Hz, 1H), 4.28-4.17 (m, 3H), 3.32 (q, J=7.0 Hz, 2H), 3.23 (t, J=5.7 Hz, 2H), 2.70 (t, J=6.4 Hz, 2H), 2.52 (d, J=7.2 Hz, 2H), 1.98-1.82 (m, 3H), 1.68-1.56 (m, 2H), 1.50 (s, 2H), 1.45-1.32 (m, 4H), 1.12 (t, J=7.0 Hz, 3H), 0.90 (d, J=6.7 Hz, 7H).

¹³C NMR (101 MHz, CDCl₃) δ 144.29, 142.87, 141.05, 132.97, 128.70, 128.48, 127.70, 126.34, 125.18, 124.63, 122.53, 110.42, 59.60, 48.49, 47.35, 45.51, 45.25, 30.30, 29.34, 28.30, 23.54, 22.46, 22.36, 10.93.

### N-cyclopentyl-3-(cyclopentylmethyl)-N-((1-ethyl-1,2,3,4-tetrahydroquinolin-6-yl)-methyl)benzenesulfonamide (Compound 15)

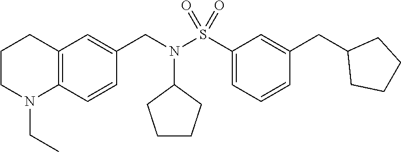 [SMILES: CCN1CCCc2cc(CN(C3CCCC3)S(=O)(=O)c3cccc(CC4CCCC4)c3)ccc21]

Purification (Hexane/EtOAc 95:5). Yield=41%. LC-MS: t^{R} 17.22 min, (analytical HPLC, 10 to 90% acetonitrile (0.1% TFA) in water (0.1% TFA) over 20 min, flow rate of 1.0 mL/min). HRMS calculated: 481.28832 for C₂₉H₄₁N₂O₂S [M+H]⁺, found: 481.28808.

¹H NMR (400 MHz, CDCl₃) δ 7.71 (d, J=7.8 Hz, 2H), 7.31 (d, J=7.9 Hz, 2H), 6.98 (d, J=8.4 Hz, 1H), 6.90 (s, 1H), 6.50 (d, J=8.4 Hz, 1H), 4.33-4.06 (m, 3H), 3.61 (t, J=6.7 Hz, 2H), 3.41-3.27 (m, 5H), 3.22 (t, J=5.6 Hz, 2H), 2.93 (t, J=6.8 Hz, 2H), 2.69 (t, J=6.4 Hz, 2H), 1.92 (p, J=6.1 Hz, 2H), 1.69-1.46 (m, 4H), 1.43-1.30 (m, 4H), 1.11 (t, J=7.0 Hz, 3H).

¹³C NMR (101 MHz, CDCl₃) δ 144.17, 143.91, 139.03, 129.38, 128.36, 127.31, 126.18, 125.15, 122.48, 110.38, 72.92, 59.52, 58.87, 48.42, 47.27, 45.45, 36.11, 29.31, 28.24, 23.51, 22.39, 10.81.

### Example 4

### A) Preparation of N-cyclopentyl-N-((1-ethyl-1,2,3,4-tetrahydroquinolin-6-yl)methyl)-3-(pyridin-2-ylamino)benzenesulfonamide (Compound 16)

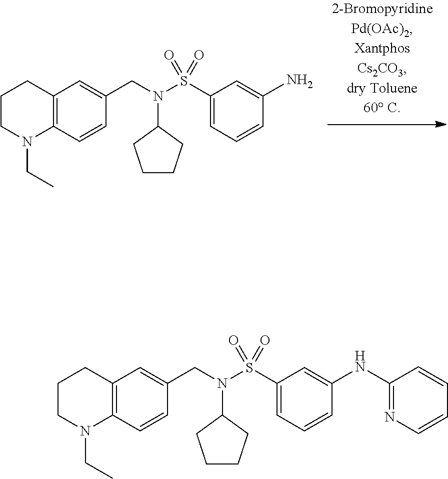 [SMILES: CCN1CCCc2cc(CN(C3CCCC3)S(=O)(=O)c3cccc(N)c3)ccc21 ; CCN1CCCc2cc(CN(C3CCCC3)S(=O)(=O)c3cccc(Nc4ccccn4)c3)ccc21]

3-Amino-N-cyclopentyl-N-((1-ethyl-1,2,3,4-tetrahydroquinolin-6-yl)methyl)benzene-sulfonamide (71 mg, 0.172 mmol, 1 eq), 2-bromopyridine (18 μL, 0.189 mmol, 1.1 eq) and Cs₂CO₃ (112 mg, 0.344 mmol, 2 eq) were charged in a vial in the presence of anhydrous toluene (1 mL) as solvent. The mixture was stirred at room temperature while different cycles of vacuum and Argon flush were carried out. A solution of Pd(OAc)₂ (3 mg, 0.014, 8% mol) and 4,5-Bis(diphenylphosphino)-9,9-dimethylxanthene (Xantphos) (16 mg, 0.028 mmol, 16% mol) in anhydrous toluene was added under Argon atmosphere and the resulting solution was heated at 60° C. After one hour the reaction was complete and the mixture was partitioned between EtOAc and water. The organic phase was washed twice with water and then the combined aqueous layers were counter extracted with one aliquot of EtOAc. The organic layers were collected, dried over anhydrous sodium sulfate, filtered and volatiles evaporated under vacuum to obtain a crude which was purified by column chromatography (Hex/EtOAc 8:2). The title compound was obtained as a pale-yellow solid (25 mg, 30%).

LC-MS: t^{R} 14.77 min, (analytical HPLC, 10 to 90% acetonitrile (0.1% TFA) in water (0.1% TFA) over 20 min, flow rate of 1.0 mL/min). HRMS calculated: 491.24752 for C₂₈H₃₅N₄O₂S [M+H]⁺, found: 491.24727. Calculated: 513,23002 for C₂₈H₃₄N₄NaO₂S [M+H]⁺, found: 513,22925.

¹H NMR (400 MHz, CDCl₃) δ 8.22 (dd, J=5.0, 1.9 Hz, 1H), 7.78 (d, J=2.1 Hz, 1H), 7.69 (dt, J=7.2, 2.2 Hz, 1H), 7.57-7.46 (m, 1H), 7.43-7.32 (m, 2H), 7.00-6.93 (m, 1H), 6.93-6.83 (m, 2H), 6.83-6.75 (m, 2H), 6.49 (d, J=8.4 Hz, 1H), 4.26 (s, 2H), 4.21-4.15 (m, 1H), 3.30 (q, J=7.0 Hz, 2H), 3.21 (t, J=5.6 Hz, 2H), 2.67 (t, J=6.4 Hz, 2H), 1.90 (p, J=6.1 Hz, 2H), 1.71-1.61 (m, 2H), 1.61-1.47 (m, 2H), 1.47-1.35 (m, 4H), 1.10 (t, J=7.0 Hz, 3H).

¹³C NMR (101 MHz, CDCl₃) δ 155.13, 148.23, 144.29, 142.00, 141.58, 137.91, 129.73, 128.55, 126.42, 125.10, 122.54, 122.31, 120.17, 117.43, 115.96, 110.42, 109.87, 59.75, 48.46, 47.49, 45.50, 29.47, 28.28, 23.56, 22.43, 10.91.

### Preparation of N-cyclopentyl-N-((1-ethyl-1,2,3,4-tetrahydroquinolin-6-yl)methyl)-4-(pyridin-2-ylamino)benzenesulfonamide (Compound 28)

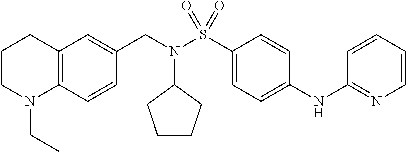 [SMILES: CCN1CCCc2cc(CN(C3CCCC3)S(=O)(=O)c3ccc(Nc4ccccn4)cc3)ccc21]

Purification (Hexane/EtOAc 7:3). Yield=57%. LC-MS: t^{R} 14.86 min, (analytical HPLC, 10 to 90% acetonitrile (0.1% TFA) in water (0.1% TFA) over 20 min, flow rate of 1.0 mL/min). HRMS calculated: 491.24752 for C₂₈H₃₅N₄O₂S [M+H]⁺, found: 491.24766. Calculated: 513,23002 for C₂₈H₃₄N₄NaO₂S [M+H]⁺, found: 513,23019.

¹H NMR (400 MHz, CDCl₃) δ 8.22 (dd, J=5.0, 1.9 Hz, 1H), 7.78 (d, J=2.1 Hz, 1H), 7.69 (dt, J=7.2, 2.2 Hz, 1H), 7.57-7.46 (m, 1H), 7.43-7.32 (m, 2H), 7.00-6.93 (m, 1H), 6.93-6.83 (m, 2H), 6.83-6.75 (m, 2H), 6.49 (d, J=8.4 Hz, 1H), 4.26 (s, 2H), 4.21-4.15 (m, 1H), 3.30 (q, J=7.0 Hz, 2H), 3.21 (t, J=5.6 Hz, 2H), 2.67 (t, J=6.4 Hz, 2H), 1.90 (p, J=6.1 Hz, 2H), 1.71-1.61 (m, 2H), 1.61-1.47 (m, 2H), 1.47-1.35 (m, 4H), 1.10 (t, J=7.0 Hz, 3H).

¹³C NMR (101 MHz, CDCl₃) δ 155.13, 148.23, 144.29, 142.00, 141.58, 137.91, 129.73, 128.55, 126.42, 125.10, 122.54, 122.31, 120.17, 117.43, 115.96, 110.42, 109.87, 59.75, 48.46, 47.49, 45.50, 29.47, 28.28, 23.56, 22.43, 10.91.

### Example 5

### A) Preparation of 3-((bis(dimethylamino)methylene)amino)-N-cyclopentyl-N-((1-ethyl-1,2,3,4-tetrahydroquinolin-6-yl)methyl)benzenesulfonamide (Compound 23)

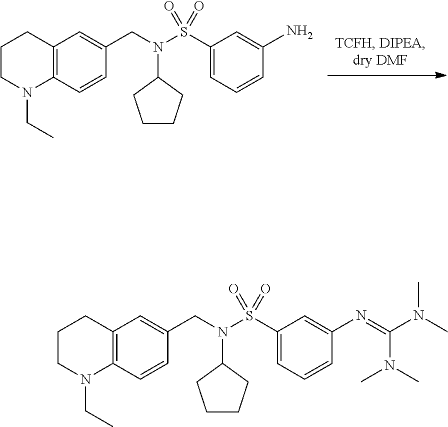 [SMILES: CCN1CCCc2cc(CN(C3CCCC3)S(=O)(=O)c3cccc(N)c3)ccc21 ; CCN1CCCc2cc(CN(C3CCCC3)S(=O)(=O)c3cccc(N=C(N(C)C)N(C)C)c3)ccc21]

To a stirring solution of 4-amino-N-cyclopentyl-N-((1-ethyl-1,2,3,4-tetrahydroquinolin-6-yl)methyl)benzenesulfona-mide (50 mg, 0.121 mmol, 1 eq) and DIPEA (63 μL, 0.364 mmol, 3 eq) in anhydrous DMF (1.3 mL), N,N,N′,N′-tetramethylchloroformamidinium hexafluorophosphate (TCFH) (68 mg, 0.242 mmol, 2 eq) was added and the reaction stirred at room temperature overnight. The mixture was extracted in EtOAc and washed with NaHCO₃ sat.sol. twice and water. The aqueous solutions were counter extracted with one aliquot of EtOAc. The organic phases were collected, dried over anhydrous sodium sulfate, filtered and volatiles evaporated under vacuum to obtain a crude mixture which was purified by column chromatography (DCM/MeOH form 99:1 to 97:3, plus additive 1% NH₃). The product was obtained as an off-white solid (53 mg, 90%).

LC-MS: t^{R} 14.73 min, (analytical HPLC, 10 to 90% acetonitrile (0.1% TFA) in water (0.1% TFA) over 20 min, flow rate of 1.0 mL/min). HRMS calculated: 512.30537 for C₂₈H₄₂N₅O₂S [M+H]⁺, found: 512.30542.

¹H NMR (400 MHz, CDCl₃) δ 7.33-7.25 (m, 2H), 7.09 (d, J=2.2 Hz, 1H), 6.99 (dd, J=8.3, 2.2 Hz, 1H), 6.96-6.88 (m, 2H), 6.50 (d, J=8.3 Hz, 1H), 4.28-4.14 (m, 3H), 3.30 (q, J=7.0 Hz, 2H), 3.21 (t, J=5.6 Hz, 2H), 2.71 (s, 14H), 1.92 (p, J=6.1 Hz, 2H), 1.65-1.54 (m, 2H), 1.53-1.43 (m, 2H), 1.43-1.31 (m, 4H), 1.10 (t, J=7.0 Hz, 3H). ¹³C NMR (101 MHz, CDCl₃) b 164.40, 160.81, 144.24, 141.52, 129.39, 128.38, 126.18, 125.68, 125.61, 122.56, 119.85, 118.49, 110.45, 59.56, 48.48, 47.49, 45.51, 39.84, 29.31, 28.30, 23.51, 22.47, 10.91.

### 4-((bis(dimethylamino)methylene)amino)-N-cyclopentyl-N-((1-ethyl-1,2,3,4-tetrahydroquinolin-6-yl)methyl)benzenesulfonamide (Compound 29)

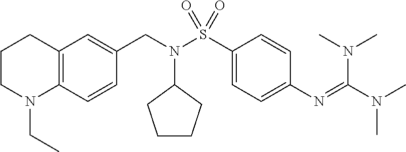 [SMILES: CCN1CCCc2cc(CN(C3CCCC3)S(=O)(=O)c3ccc(N=C(N(C)C)N(C)C)cc3)ccc21]

Purification (DCM/MeOH 99:1 to 97:3, additive 1% NH₃). Yield=63%. LC-MS: t^{R}14.80 min, (analytical HPLC, 10 to 90% acetonitrile (0.1% TFA) in water (0.1% TFA) over 20 min, flow rate of 1.0 mL/min). HRMS calculated: 512.30537 for C₂₈H₄₂N₅O₂S [M+H]⁺, found: 512.30522.

¹H NMR (400 MHz, CDCl₃) δ 7.62 (d, J=8.3 Hz, 2H), 6.98 (d, J=8.4 Hz, 1H), 6.92 (s, 1H), 6.69 (d, J=8.2 Hz, 2H), 6.50 (d, J=8.4 Hz, 1H), 4.26-4.09 (m, 3H), 3.30 (q, J=7.1 Hz, 2H), 3.21 (t, J=5.6 Hz, 2H), 2.76-2.62 (m, 14H), 1.91 (p, J=6.1 Hz, 2H), 1.64-1.52 (m, 2H), 1.51-1.42 (m, 2H), 1.35 (d, J=5.0 Hz, 4H), 1.10 (t, J=7.0 Hz, 3H).

¹³C NMR (101 MHz, CDCl₃) δ 160.86, 156.07, 144.09, 130.96, 128.49, 128.26, 126.05, 125.76, 122.44, 121.37, 110.39, 59.35, 48.41, 47.15, 45.44, 39.80, 29.19, 28.24, 23.57, 22.42, 10.85.

### Example 6

### A) Preparation of 4-(((1H-imidazol-4-yl)methyl)amino)-N-cyclopentyl-N-((1-ethyl-1,2,3,4-tetrahydroquinolin-6-yl)methyl)benzenesulfonamide (Compound 32)

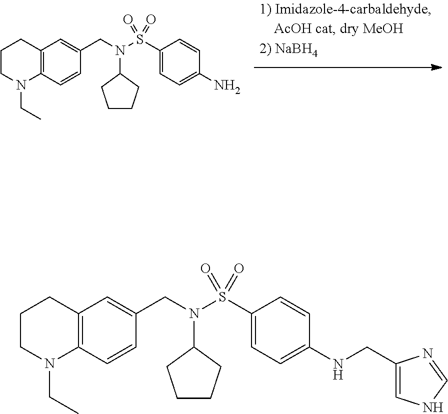 [SMILES: CCN1CCCc2cc(CN(C3CCCC3)S(=O)(=O)c3ccc(N)cc3)ccc21 ; CCN1CCCc2cc(CN(C3CCCC3)S(=O)(=O)c3ccc(NCc4c[nH]cn4)cc3)ccc21]

To a stirring solution of 4-amino-N-cyclopentyl-N-((1-ethyl-1,2,3,4-tetrahydroquinolin-6-yl)methyl)benzenesulfonamide (60 mg, 0.145 mmol, 1 eq) in anhydrous MeOH (2 mL), imidazole-4-carbaldehyde (42 mg, 0.434 mmol, 3 eq) and a catalytic amount of acetic acid was added (5 μL). The reaction was stirred under inert atmosphere overnight until complete imine formation was observed. Then NaBH₄ (33 mg, 0.87 mmol, 6 eq) was added and the reaction allowed to stir at room temperature for 12 hours when the conversion was complete. The reaction was extracted in EtOAc and washed with water twice, then aqueous phases were counter extracted with one aliquot of EtOAc. The organic phases were collected, dried over anhydrous sodium sulfate, filtered and volatiles evaporated under reduced pressure. The crude mixture was purified by flash chromatography (DCM/MeOH 98:2 to 9:1) to afford a white solid (54 mg, 76%).

LC-MS: t^{R} 11.96 min, (analytical HPLC, 10 to 90% acetonitrile (0.1% TFA) in water (0.1% TFA) over 20 min, flow rate of 1.0 mL/min). HRMS calculated: 494.25842 for C₂₇H₃₆N₅O₂S [M+H]⁺, found: 494.25878. Calculated: 511.26171 for C₂₇H₃₇N₅O₃S [M+2H]²+/2, found: 511.26202.

¹H NMR (400 MHz, CDCl₃) δ 11.36 (s, 1H), 7.66 (s, 1H), 7.50 (d, J=8.4 Hz, 2H), 7.00 (dd, J=8.5, 2.3 Hz, 1H), 6.88 (s, 1H), 6.72 (s, 1H), 6.56 (d, J=8.5 Hz, 2H), 6.52 (d, J=8.5 Hz, 1H), 5.15 (t, J=6.5 Hz, 1H), 4.45 (d, J=6.3 Hz, 2H), 4.24-4.09 (m, 3H), 3.31 (q, J=7.0 Hz, 2H), 3.23 (t, J=5.6 Hz, 2H), 2.68 (t, J=6.4 Hz, 2H), 1.91 (p, J=6.0 Hz, 2H), 1.68-1.58 (m, 2H), 1.58-1.46 (m, 2H), 1.42-1.31 (m, 4H), 1.11 (t, J=7.0 Hz, 3H).

¹³C NMR (101 MHz, CDCl₃) δ 150.53, 144.31, 136.33, 134.90, 129.14, 127.96, 127.67, 125.85, 125.52, 122.70, 116.56, 112.42, 110.58, 59.55, 48.44, 47.08, 45.52, 38.11, 29.41, 28.30, 23.74, 22.36, 10.91.

### 4-((2-bromobenzyl)amino)-N-cyclopentyl-N-((1-ethyl-1,2,3,4-tetrahydroquinolin-6-yl)methyl)benzenesulfonamide (Compound 31)

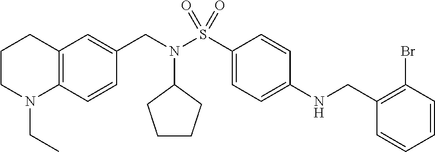 [SMILES: CCN1CCCc2cc(CN(C3CCCC3)S(=O)(=O)c3ccc(NCc4ccccc4Br)cc3)ccc21]

Purification (Hexane/EtOAc 85:15). Yield=48%. LC-MS: t^{R} 12.50 min, (analytical HPLC, 10 to 90% acetonitrile (0.1% TFA) in water (0.1% TFA) over 20 min, flow rate of 1.0 mL/min). HRMS calculated: 582.17844 for C₃₀H₃₇BrN₃O₂S [M+H]⁺, found: 582.17832. Calculated: 584.17639 for C₃₀H₃₇BrN₃O₂S [M+H]⁺, found: 584.17625.

¹H NMR (400 MHz, CDCl₃) δ 7.60-7.52 (m, 3H), 7.32 (d, J=7.7 Hz, 1H), 7.25 (t, J=7.4 Hz, 1H), 7.14 (t, J=7.6 Hz, 1H), 6.99 (d, J=8.4 Hz, 1H), 6.91 (s, 1H), 6.55 (d, J=8.3 Hz, 2H), 6.51 (d, J=8.4 Hz, 1H), 4.78 (s, 1H), 4.42 (s, 2H), 4.23-4.13 (m, 3H), 3.31 (q, J=7.1 Hz, 2H), 3.21 (t, J=5.7 Hz, 2H), 2.69 (t, J=6.5 Hz, 2H), 1.92 (p, J=6.0 Hz, 2H), 1.67-1.55 (m, 2H), 1.54-1.44 (m, 3H), 1.44-1.32 (m, 4H), 1.11 (t, J=7.0 Hz, 3H).

¹³C NMR (101 MHz, CDCl₃) δ 150.81, 144.11, 137.10, 133.03, 129.20, 129.09, 129.03, 128.63, 128.31, 127.75, 126.10, 125.76, 123.30, 122.51, 112.05, 110.47, 59.41, 48.43, 47.89, 47.17, 45.50, 29.30, 28.25, 23.62, 22.43, 10.88.

### Example 7

### Activity of Compounds 1, 2, 14, and 32

U2OS cells were treated with compounds 1, 2, 14, and 32 at concentrations of 0, 0.625, 1.25, 2.5, 5, 10, 15, 20 and 25 μM. Cell proliferation (confluency) and cytotoxicity (CellTox™ Green confluency) were assessed using the IncuCyte® live cell imaging platform. Cell death was calculated as a percentage of overall confluency, using CellTox™ Green confluency as a percentage of total cell confluence. Cell toxicity measurements were capped at the time point at which cells reached complete confluency. This was to account for cell death due to over-confluency of cells, rather than as a result of compounds' effects. The same experiments were also performed on a list of ALT-positive, telomerase-positive, ALT- and telomerase-positive, and normal cell lines. For the ALT-positive cell lines GM847, SK—N—FI, HuO9, Saos-2, CAL72, IIICF/c, and HS729 were employed. For the telomerase-positive cells HCT116, HeLa, HT1080, SK—N—Be2, and SK—N-AS, and KELLY were used. For the ALT-positive/telomerase-positive and mortal cell strains GM847 hTERT, MRC-5, and IMR-90 were employed.

To determine whether the compounds disrupt the FANCM-BTR interaction, co-immunoprecipitation experiments using the RMI1 antibody were performed in the presence and absence of compounds 1, 2, 14, 32, and the already reported compound PIP-199 treatments as well as the scambled, siFANCM and DMSO. This method provides information indicating the direct binding of proteins and is highly specific and simple to perform. U2OS cells were seeded at 50% confluency in 150 cm² flasks (BD Falcon). After 24 hours, cells were treated with scrambled, siFANCM and DMSO (0.3%, determined by the maximal DMSO concentration present in drugs) treatments, and 2, 14, 32, PIP-199 treatments at their IC₅₀ concentrations. After 72 hours, cells were washed once with warm PBS, trypsinised for 5 minutes at 37° C., resuspended in media and counted using a Beckman Coulter cell counter. Cells were collected in pellets in Protein Lobind tubes (Eppendorf) by centrifugation at 200×g at 4° C. and supernatant was discarded. Fresh pellets were lysed using Lysis Buffer B (20 mM HEPES-KOH pH 7.9, 200 mM NaCl, 2 mM MgCl2, 10% v/v Glycerol, 0.1 v/v Triton X-100) with 1 mM PMSF (Sigma), 1 mM DTT (Sigma) and 1×Complete® Protease inhibitor cocktail (Roche), then incubated at 4° C. on a rotating rack for 1 hour. Lysates were centrifuged at 13,000 rpm at 4° C. for 40 minutes, and supernatants were collected. Protein G Dynabeads® (Life Technologies) were prepared by washing beads twice with 1×PBST. Dynabeads® were resuspended in anti-RMI1 antibody (Rb, Proteintech: 14630-1-AP, 0.25 mg/mL) at a concentration of 0.8 μg/mg lysate. 5% lysates were used. Antibodies and Dynabeads® were incubated with rotation for 10 minutes at room temperature, tubes were placed on a magnetic holder and supernatant was removed to remove unbound antibody. Antibody-bound beads were then washed once in 1×PBST. Supernatant was removed from the cell pellet and the lysate was added to the antibody-bound beads, then incubated on a rotator at 4° C. overnight. The next day, beads were separated on a magnetic holder, with supernatants (flow-through) transferred to clean tubes for subsequent analysis of immunoprecipitation efficiency.

The Dynabeads®-Ab-protein complex was washed three times using 200 μL ice cold Lysis Buffer B for each wash. Beads were separated on a magnetic holder between each wash, supernatant was removed, and beads were resuspended by gentle pipetting. 1×LDS buffer was added to Dynabeads®-Ab-protein complex, then heated for 10 minutes at 70° C. On a magnetic holder, the eluates were transferred to fresh tubes. The eluates were analysed by western blot and the data was quantitated using ImageJ software (National Institutes of Health). Eluates were expressed as a percentage of their respective lysates to show relative differences between treatments.

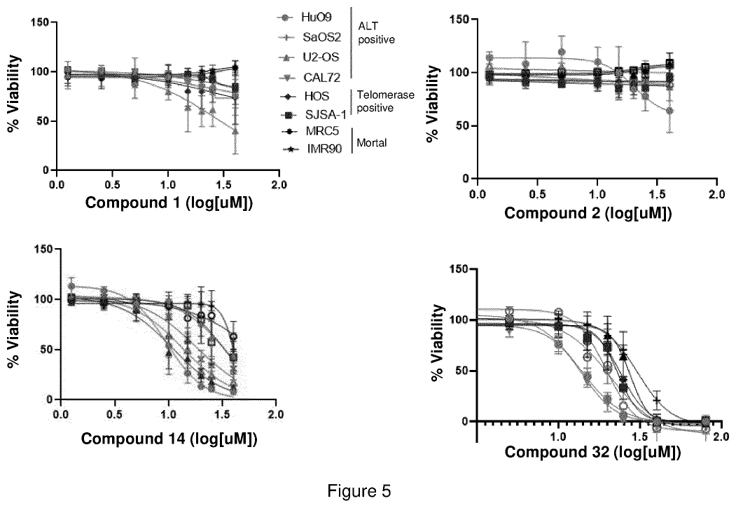

Preliminary cell-based assays indicated that the best compound 32 shows inhibition of U2OS cell viability and promising lead-like properties (i.e. fulfills all the Lipinski's rule of five). The IC₅₀ (i.e., the concentration of drug that inhibits 50% of cell growth) and EC₅₀ values (i.e., the concentration that effectively causes 50% of cell death) for compound 32 on U2OS were 15.52 μM and 1.36 μM, respectively (FIG. 6a). Moreover, a comparison of IC₅₀ and EC₅₀ values across the panel of cell lines (FIG. 6b) treated with compound 32 demonstrated that this compound shows selective induction of ALT-cell death. Additionally, compounds 1 and 14 are causing a concentration-dependent toxicity that is partly selective to the ALT cell lines (FIG. 5).

Finally, co-immunoprecipitation experiments on the ALT cancer cell line U2OS using an antibody against RMI1 (of the BTR complex) demonstrated that compound 32 treatment did cause a decrease in FANCM levels at the IC₅₀ concentration in U2OS cells (FIGS. 6c and 6d). This suggested that compound 32 and its analogues described herein are directly inhibiting the FANCM/BTR interaction. Most interestingly, the FANCM protein levels in the input were quantified suggesting that compound 32 by binding the BTR complex might also induce the FANCM degradation.

1. A tetrahydroquinoline compound of Formula (I) or a pharmaceutically acceptable salt thereof 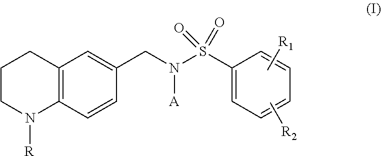
wherein
A is (C₃-C₇)cycloalkyl; (C₃-C₇)heterocycloalkyl comprising from 1 to 3 heteroatoms selected from O, N, and S; (C₁-C₇)alkyl, (C₁-C₇)heteroalkyl comprising from 1 to 3 heteroatoms selected from O, N, and S; an optionally substituted phenyl ring or an optionally substituted (5-9)-membered heteroaromatic ring comprising from 1 to 3 heteroatoms selected from O, N, and S;
R is H, (C₁-C₄)alkyl; (C₁-C₃)alkoxy(C₁-C₃)alkyl, (C₃-C₇)cycloalkyl;
R₁ and R₂ are, independently from each other, selected from the group consisting of H, NO₂, CN, halogen, (C₁-C₄)alkyl; (C₂-C₄)alkenyl; (C₁-C₄)alkoxy(C₁-C₄)alkyl, (C₃-C₇)cycloalkyl(C₁-C₄)alkyl, (C₁-C₄)alkyl(5-9)-membered heteroaromatic ring comprising from 1 to 3 heteroatoms selected from O, N, and S,
amino optionally substituted with bis(dimethylamino)methylene, pyridyl, trifluoromethyl-SO₂—, (C₁-C₃)alkyl-SO₂—,
a group NH—CO—R₃,
wherein R₃ is selected from the group consisting of benzyl, (C₃-C₇)cycloalkyl(C₁-C₄)alkyl, phenyl, vinyl, halogen-CH₂—, (C₁-C₃)alkyl;
a group NH—CH₂—R₄
wherein R₄ is an optionally substituted phenyl ring or an optionally substituted (5-9)-membered heteroaromatic ring comprising from 1 to 3 heteroatoms selected from O, N, and S; or
R₁ and R₂ together form an optionally substituted aromatic ring comprising 6 carbon atoms.

2. The tetrahydroquinoline compound of claim 1, wherein R₁ or R₂ is a group NH—CH₂—R₄ wherein R₄ is an optionally substituted aromatic ring or an optionally substituted (5-9)-membered heteroaromatic ring comprising from 1 to 3 heteroatoms selected from O, N, and S.

3. The tetrahydroquinoline compound of claim 1, wherein A is (C₃-C₇)cycloalkyl, preferably A is cyclopentyl.

4. The tetrahydroquinoline compound of claim 1, wherein A is (C₃-C₇)cycloalkyl, preferably selected from the group consisting of cyclopropyl, cyclobutyl, cyclopentyl, cyclohexyl and cycloheptyl, more preferably A is cyclopentyl.

5. The tetrahydroquinoline compound of claim 1, wherein A is (C₁-C₇)alkyl, preferably selected from the group consisting of methyl, ethyl, propyl, butyl, pentyl, hexyl, propan-2-yl, butan-2-yl, pentan-2-yl, hexan-2-yl, pentan-3-yl, 2-methylpentan-3-yl, 2,4-dimethylpentan-3-yl, tert-butyl, 2-methylbutyl, 3-methylbutyl, 2-metylpentyl, 3-metylpentyl and 4-metylpentyl.

6. The tetrahydroquinoline compound of claim 1, wherein A is of (5-9)-membered heteroaromatic ring comprising from 1 to 3 heteroatoms selected from O, N and S, preferably selected from the group consisting of pyrrolyl, pyrrol-2-yl, pyrrol-3-yl, furan-2-yl, furan-3-yl, thiophen-2-yl, thiophen-3-yl, imidazol-4-yl, 1,3-oxazol-5-yl, 1,3-oxazol-4-yl, 1,3-thiazol-5-yl, 1,3-thiazol-4-yl, 1,2,3-triazol-4-yl, 1,2,4-triazol-5-yl, pyridin-2-yl, pyridin-3-yl, pyridin-4-yl, pyrimidin-4-yl, pyrimidin-2-yl, pyrimidin-5-yl, pyrazin-2-yl, indol-2-yl, indol-3-yl, isoindol-1-yl, 1-benzofuran-3-yl, 1-benzofuran-2-yl, 2-benzofuran-1-yl, 2-benzothiophen-1-yl, 1-benzothiophen-3-yl, 2-benzothiophen-3-yl and benzimidazol-2-yl.

7. The tetrahydroquinoline compound of claim 1, wherein R is (C₁-C₄) alkyl, preferably it is ethyl.

8. The tetrahydroquinoline compound of claim 1, wherein one of R₁ or R₂ is H.

9. The tetrahydroquinoline compound of claim 1, wherein one of R₁ and R₂ is H and the other is a group NH—CH₂—R₄ wherein R₄ is an optionally substituted phenyl ring or an optionally substituted (5-9)-membered heteroaromatic ring comprising from 1 to 3 heteroatoms selected from O, N, and S.

10. The tetrahydroquinoline compound of claim 9, wherein R₄ is selected from the group consisting of pyrrol-2-yl, pyrrol-3-yl, furan-2-yl, furan-3-yl, thiophen-2-yl, thiophen-3-yl, imidazol-4-yl, 1,3-oxazol-5-yl, 1,3-oxazol-4-yl, 1,3-thiazol-5-yl, 1,3-thiazol-4-yl, 1,2,3-triazol-4-yl, 1,2,4-triazol-5-yl, pyridin-2-yl, pyridin-3-yl, pyridin-4-yl, pyrimidin-4-yl, pyrimidin-2-yl, pyrimidin-5-yl, pyrazin-2-yl, indol-2-yl, indol-3-yl, isoindol-1-yl, 1-benzofuran-3-yl, 1-benzofuran-2-yl, 2-benzofuran-1-yl, 2-benzothiophen-1-yl, 1-benzothiophen-3-yl, 2-benzothiophen-3-yl, benzimidazol-2-yl.

11. The tetrahydroquinoline compound of claim 10, wherein R₄ is imidazol-4-yl.

12. The tetrahydroquinoline compound of claim 9, wherein R₄ is phenyl substituted with halogen, e preferably with bromine.

13. The tetrahydroquinoline compound of claim 1, wherein, when R₁ or R₂ is halogen, it is bromine.

14. The tetrahydroquinoline compound of claim 1, wherein when R₁ or R₂ is (C₁-C₄)alkyl, it is selected from butan-2-yl (or sec-butyl) and propan-2-yl (or isobutyl).

15. The tetrahydroquinoline compound of claim 1, wherein when R₁ or R₂ is (C₂-C₄)alkenyl, it is p vinyl.

16. The tetrahydroquinoline compound of claim 1, wherein when R₁ or R₂ is (C₁-C₄)alkoxy(C1-C4)alkyl, it is methoxyethyl.

17. The tetrahydroquinoline compound of claim 1, wherein when R₁ or R₂ is (C₁-C₄)alkyl(5-9)-membered heteroaromatic ring comprising from 1 to 3 heteroatoms selected from O, N, and S, it is furan-2-ylmethyl.

18. The tetrahydroquinoline compound of claim 1, wherein when R₁ or R₂ is (C₃-C₇)cycloalkyl(C₁-C₄)alkyl, it is 3-(cyclopentyl)methyl.

19. The tetrahydroquinoline compound of claim 1, wherein, when R₁ or R₂ is amino optionally substituted with bis(dimethylamino)methylene, pyridyl, trifluoromethyl-SO₂—, (C₁-C₃) alkyl-SO₂—, it is selected from amino substituted with bis(dimethylamino)methylene, pyridyl, trifluoromethyl-SO₂—, methyl-SO₂—.

20. The tetrahydroquinoline compound of claim 1, wherein R₁ or R₂ is a group NH—CO—R₃, and R₃ is selected from the group consisting of benzyl, (C₃-C₇)cycloalkyl(C₁-C₄)alkyl, phenyl, vinyl, halogen-CH₂—, (C₁-C₃)alkyl, preferably R₃ is selected from the group consisting of benzyl, cyclopentylethyl, phenyl, vinyl, bromo-CH₂—, ethyl.

21. The tetrahydroquinoline compound of claim 1, wherein R₁ and R₂ together also form an optionally substituted aromatic ring comprising 6 carbon atoms.

22. The tetrahydroquinoline compound of claim 1, wherein the compound of formula (I) is selected from the group consisting of
N-cyclopentyl-N-((1-ethyl-1,2,3,4-tetrahydroquinolin-6-yl)methyl)-3-nitrobenzenesulfonamide (Compound 1);
N-cyclopentyl-N-((1-ethyl-1,2,3,4-tetrahydroquinolin-6-yl)methyl)-4-nitrobenzenesulfonamide (Compound 2);
3-Bromo-N-cyclopentyl-N-((1-ethyl-1,2,3,4-tetrahydroquinolin-6-yl)methyl)benzenesulfonamide (Compound 3);
N-cyclopentyl-N-((1-ethyl-1,2,3,4-tetrahydroquinolin-6-yl)methyl)-4-vinylbenzenesulfonamide (Compound 4);
N-cyclopentyl-N-((1-ethyl-1,2,3,4-tetrahydroquinolin-6-yl)methyl)-3-isopropylbenzenesulfonamide (Compound 5);
N-cyclopentyl-N-((1-ethyl-1,2,3,4-tetrahydroquinolin-6-yl)methyl)naphthalene-2-sulfonamide (Compound 6);
N-cyclopentyl-N-((1-ethyl-1,2,3,4-tetrahydroquinolin-6-yl)methyl)-3-isobutylbenzenesulfonamide (Compound 7);
N-(4-(N-cyclopentyl-N-((1-ethyl-1,2,3,4-tetrahydroquinolin-6-yl)methyl)sulfamoyl)phenyl)-2-phenylacetamide (Compound 8);
3-cyclopentyl-N-(4-(N-cyclopentyl-N-((1-ethyl-1,2,3,4-tetrahydroquinolin-6-yl)methyl)sulfamoyl)phenyl)propenamide (Compound 9);
N-cyclopentyl-N-((1-ethyl-1,2,3,4-tetrahydroquinolin-6-yl)methyl)-3-(furan-2-ylmethyl)benzenesulfonamide (Compound 10);
N-(3-(N-cyclopentyl-N-((1-ethyl-1,2,3,4-tetrahydroquinolin-6-yl)methyl)sulfamoyl)phenyl)benzamide (Compound 11);
N-(3-(N-cyclopentyl-N-((1-ethyl-1,2,3,4-tetrahydroquinolin-6-yl)methyl)sulfamoyl)phenyl)-2-phenylacetamide (Compound 12);
3-cyclopentyl-N-(3-(N-cyclopentyl-N-((1-ethyl-1,2,3,4-tetrahydroquinolin-6-yl)methyl)sulfamoyl)phenyl)propenamide (Compound 13);
N-cyclopentyl-N-((1-ethyl-1,2,3,4-tetrahydroquinolin-6-yl)methyl)-4-(2-methoxyethyl)benzenesulfonamide (Compound 14);
N-cyclopentyl-3-(cyclopentylmethyl)-N-((1-ethyl-1,2,3,4-tetrahydroquinolin-6-yl)methyl)benzenesulfonamide (Compound 15);
N-cyclopentyl-N-((1-ethyl-1,2,3,4-tetrahydroquinolin-6-yl)methyl)-3-(pyridin-2-ylamino)benzenesulfonamide (Compound 16);
N-(3-(N-cyclopentyl-N-((1-ethyl-1,2,3,4-tetrahydroquinolin-6-1)methyl)sulfamoyl)phenyl)acrylamide (Compound 17);
2-Bromo-N-(3-(N-cyclopentyl-N-((1-ethyl-1,2,3,4-tetrahydroquinolin-6-yl)methyl)sulfamoyl)phenyl)acetamide (Compound 18);
4-Cyano-N-cyclopentyl-N-((1-ethyl-1,2,3,4-tetrahydroquinolin-6-yl)methyl)benzenesulfonamide (Compound 19);
N-cyclopentyl-N-((1-ethyl-1,2,3,4-tetrahydroquinolin-6-yl)methyl)-2-nitrobenzenesulfonamide (Compound 20);
N-(3-(N-cyclopentyl-N-((1-ethyl-1,2,3,4-tetrahydroquinolin-6-yl)methyl)sulfamoyl)phenyl)propionamide (Compound 21);
N-cyclopentyl-N-((1-ethyl-1,2,3,4-tetrahydroquinolin-6-yl)methyl)-3-(methylsulfonamido)benzenesulfonamide (Compound 22);
3-((Bis(dimethylamino)methylene)amino)-N-cyclopentyl-N-((1-ethyl-1,2,3,4-tetrahydroquinolin-6-yl)methyl)benzenesulfonamide (Compound 23);
N-cyclopentyl-N-((1-ethyl-1,2,3,4-tetrahydroquinolin-6-yl)methyl)-4-(N-(methylsulfonyl)methylsulfonamido)benzenesulfonamide (Compound 24);
N-(4-(N-cyclopentyl-N-((1-ethyl-1,2,3,4-tetrahydroquinolin-6-yl)methyl)sulfamoyl)phenyl)acrylamide (Compound 25);
2-Bromo-N-(4-(N-cyclopentyl-N-((1-ethyl-1,2,3,4-tetrahydroquinolin-6-yl)methyl)sulfamoyl)phenyl)acetamide (Compound 26);
N-(4-(N-cyclopentyl-N-((1-ethyl-1,2,3,4-tetrahydroquinolin-6-yl)methyl)sulfamoyl)phenyl)propionamide (Compound 27);
N-cyclopentyl-N-((1-ethyl-1,2,3,4-tetrahydroquinolin-6-yl)methyl)-4-(pyridin-2-ylamino)benzenesulfonamide (Compound 28);
4-((Bis(dimethylamino)methylene)amino)-N-cyclopentyl-N-((1-ethyl-1,2,3,4-tetrahydroquinolin-6-yl)methyl)benzenesulfonamide (Compound 29);
N-cyclopentyl-N-((1-ethyl-1,2,3,4-tetrahydroquinolin-6-yl)methyl)-4-(trifluoromethylsulfonamido)benzenesulfonamide (Compound 30);
4-((2-Bromobenzyl)amino)-N-cyclopentyl-N-((1-ethyl-1,2,3,4-tetrahydroquinolin-6-yl)methyl)benzenesulfonamide (Compound 31);
4-(((1H-imidazol-4-yl)methyl)amino)-N-cyclopentyl-N-((1-ethyl-1,2,3,4-tetrahydroquinolin-6-yl)methyl)benzenesulfonamide (Compound 32).

23. The tetrahydroquinoline compound of claim 22, wherein the compound is 4-(((1H-imidazol-4-yl)methyl)amino)-N-cyclopentyl-N-((1-ethyl-1,2,3,4-tetrahydroquinolin-6-yl)methyl)benzenesulfonamide (Compound 32).

24. A pharmaceutical composition comprising the tetrahydroquinoline compound of claim 1 and at least one pharmaceutically acceptable excipient.

25-27. (canceled)

28. A method for hampering the FANCM localization to telomeres comprising the step of administering a tetrahydroquinoline compound of claim 1 or a pharmaceutically acceptable salt thereof as a disruptor of the FANCM/BTR interaction.

29. The method of claim 28, wherein the tetrahydroquinoline compound or pharmaceutically acceptable salt thereof is used in the treatment of tumors.
